(19) United States
(12) Patent Application Publication
(10) Pub. No.: US 2026/0306416 A1
(43) Pub. Date: Oct. 8, 2026

(54) EXTRACELLULAR VESICLES FOR THE DELIVERY OF PAYLOADS TO EUKARYOTIC CELLS

(71) Applicant: ALAYA.BIO INC., Cambridge, MA (US)

(72) Inventors: Cecile BAUCHE, Paris (FR); Renaud VAILLANT, Gentilly (FR); Rachel PACHERIE, Sceaux (FR); Frederic MOURLANE, Nice (FR); Celine JAUDOUIN, Montrouge (FR)

(73) Assignee: ALAYA.BIO INC., Cambridge, MA (US)

(21) Appl. No.: 19/483,291

(22) PCT Filed: May 10, 2024
(86) PCT No.: PCT/US2024/028766

§ 371 Date: Nov. 11, 2025

Related U.S. Application Data
(60) Provisional application No. 63/501,561, filed on May 11, 2023.
(60) Provisional application No. 63/589,411, filed on Oct. 11, 2023.

(51) Int. Cl. A61K 9/50; A61K 40/31; A61K 40/42; A61K 47/69; C07K 14/705; C07K 16/28

(52) U.S. Cl. CPC A61K 9/5068; A61K 40/31; A61K 40/4211; A61K 47/6901; C07K 14/7051; C07K 16/2803; A61K 2239/13

(57) ABSTRACT

The invention relates to the production, modification, and use of extracellular vesicles (EVs), particularly for the delivery of payloads of nucleic acids. The EVs can contain retroviral capsids and can be coated with polymers. The EVs can be treated with a protease to remove proteins on the outer surface of the EV to create shaved EVs. The shaved EVs can be coated with a polymer and used for the delivery of payloads of nucleic acids to cells.

### CROSS-REFERENCE TO RELATED APPLICATIONS

This application is a U.S. National Stage Entry of PCT/US2024//028766, filed May 10, 2024, and claims priority to U.S. Provisional Application No. 63/501,561, filed May 11, 2023 and U.S. Provisional Application No. 63/589,411, filed Oct. 11, 2023, both of which are herein incorporated by reference in their entirety.

### REFERENCE TO SEQUENCE LISTING SUBMITTED ELECTRONICALLY

A Sequence Listing in XML format entitled “AYA_1_1 PCT_SL.xml”, created on May 6, 2024, and having a size of 16,524 bytes, is submitted herewith. The Sequence Listing is incorporated herein by reference. This Sequence Listing does not include any new matter and is fully supported by the disclosure as originally filed.

### BACKGROUND OF THE INVENTION

Extracellular vesicles (EVs) are vesicles secreted by many different types of cells. See e.g., Du et al., Pharmaceutics 2022, 14:2236. EVs are phospholipid-based bilayer membrane particles. EVs can be endosome-derived (“exosomes”), typically with a diameter of ~30 nm to ~150 nm. Alternatively, EVs can be generated by membrane outward budding (“ectosomes” or “microvesicles”), typically with a diameter of ~50 nm to ~1000 nm. See e.g., Teng and Fussenegger, Advanced Science 2021, 8:2003505.

EVs serve as natural vehicles for the transfer of information from one cell to another. Studies have shown that EVs can be loaded with nucleic acids, proteins, lipids and small molecules to modify recipient cells. See e.g., Joshi and Zuhorn, Materials Today Nano 2021, 100148. Thus, EVs can be used as a therapeutic tool for the clinical application of nucleic acid drugs. See e.g., Rezaie et al., Cell Communication and Signaling 2022, 20:145.

Similarly, retroviruses are phospholipid-based bilayer membrane particles. Retroviruses acquire a host-derived lipid membrane as they pass the membranes of the cell. See e.g., Rheinemann et al., Encyclopedia of Virology, 4th Edition, 2021 Elsevier Ltd., Volume 1, 519-528. All retroviruses, including lentiviruses, express a Gag polyprotein that coordinates assembly of virions. See e.g., Coffin et al., Retroviruses, Cold Spring Harbor Laboratory Press, 1997. The Gag polyprotein, when expressed alone, is released within virus-like particles. This indicates that Gag polyprotein is sufficient for assembly, envelopment, and budding of the virions. See e.g. Bell and Lever, Trends in Microbiology 2013, 21:136-44.

Retroviral, particularly lentiviral vectors, have been used as vectors to deliver payloads, including therapeutic molecules that provide direct or indirect therapeutic benefit, to mammalian cells. See e.g., Dautzenberg et al., Gene Therapy 2021, 28:89-104. For example, they have been used to generate Chimeric antigen receptor (CAR) T cells. See e.g., U.S. Pat. No. 9,102,760. Virus-like particles (VLPs) particles, which are formed by structural viral proteins that mimic the morphology of a native virus, but are non-infectious, non-replicating, and lack the genetic material, have also been used as vectors. Rheinemann et al., Encyclopedia of Virology, 4th Edition, 2021 Elsevier Ltd., Volume 1, 519-528.

The delivery of therapeutic molecules to cells holds great promise in the treatment of diseases, such as cancer. New compositions and methods are needed to increase the delivery of therapeutic molecules to cells. Applicant's invention fulfills this need.

### BRIEF SUMMARY OF THE INVENTION

The invention encompasses extracellular vesicles (EVs) and methods for making, modifying, and using them.

The invention encompasses an EV that has been treated with a protease to remove proteins on the outer surface of the EV.

In one embodiment, the EV does not have any full-length viral envelope proteins on its surface. In one embodiment, the EV does not have any full-length CAR or non-retroviral or non-lentiviral protein on its surface.

In one embodiment, the EV comprises a retroviral capsid comprising a pair of RNAs encoding a non-retroviral or non-lentiviral protein. In one embodiment, the RNA encodes a chimeric antigen receptor. In one embodiment, the capsid is an HIV-1 capsid. In one embodiment, the capsid comprises a reverse transcriptase. In one embodiment, the capsid comprises an integrase.

The EV can be coated with a polymer to form a polymeric nanoparticle (PNP). In one embodiment, the EV is coated with a cationic polymer. In one embodiment, the EV is coated with an anionic polymer. In one embodiment, the EV is coated with a zwitterionic polymer. The polymer can be selected from polyarginine, polylysine, polyether Imide (PEI), chitosan, poly(β-amino ester) (PBAE), PEI-co-polyhistidine copolymer, polylysine dendigraft/dendrimer/branched polymer, star shaped polyaminoacid based polymer, chondroitin sulfate, heparosan, poly glutamic acid (PGA), hyaluronic acid, dextran sulfate, polysarcosine, and polyethyleneglycol (PEG), polycarboxybetaine, polysulfobetaine or polyphosphorylcholine.

In one embodiment, the protease is proteinase K.

In one embodiment, the EV comprises a CD3 or NKG2D targeting agent.

In one embodiment, the EV is produced in a mammalian cell line. The invention encompasses a cell comprising an EV of the invention.

The invention encompasses a method comprising contacting the EV/PNP of the invention with a mammalian cell. In one embodiment, the cell is a human cell. In one embodiment, the cell is a T cell or an NK cell.

The invention encompasses methods of modifying the outer surface of EVs. In one embodiment, the method comprises purifying EVs from a cell; treating the EVs with a protease to remove proteins on the outer surface of the EVs; inactivating the protease; and purifying the treated EVs from the protease and from the removed extracellular proteins fragments. In one embodiment, the cell expresses retroviral Gag, Rev, and Pol proteins. In one embodiment, the EVs comprise an RNA encoding a non-retroviral or lentiviral protein. In one embodiment, the RNA encodes a chimeric antigen receptor. In one embodiment, the method comprises contacting the modified EVs/PNPs with a mammalian cell.

### BRIEF DESCRIPTION OF THE DRAWINGS

FIG. 1A-C depicts the detection by direct ELISA of human CD9 (A), human CD63 (B) and human CD81 (C) surface receptors on two unshaved EVs batches produced in HEK293-derived cells.

FIG. 2A-C depicts the simultaneous detection of human CD81/CD9 (A), human CD63/CD9 (B) and human CD63/CD81 (C) surface receptors on unshaved EVs batches produced in HEK293-derived cells by sandwich ELISA.

FIG. 3A-C depict the sandwich ELISA performed on unshaved EV and LV batches produced in HEK293-derived cells. A) titration of EV/LV using the CD81/CD9 antibody pair. B) titration of EV/LV using the CD63/CD9 antibody pair. C) titration of EV/LV using the CD63/CD81 antibody pair.

FIG. 4 shows expression levels of human receptors expressed on the surface of unshaved EV and LV batches produced in HEK293-derived cells. Expression levels were measured by flow cytometry using the MACSplex Exosome kit.

FIG. 5 depicts the Zeta potential of unshaved EV and LV batches produced in HEK293-derived cells measured by MADLS (Multiple Angle Dynamic Light Scattering).

FIG. 6 depicts the mean hydrodynamic diameter (as a measure of the size) of particles (Dh) and aggregates detected above 200 nm (Dh aggregates) of unshaved LV batches produced in HEK293-derived cells measured by MADLS (Multiple Angle Dynamic Light Scattering).

FIG. 7 depicts the mean hydrodynamic diameter (as a measure of the size) and index of polydispersity (Pdl, as a measure of size distribution) of unshaved EV batches produced in HEK293-derived cells measured by MADLS (Multiple Angle Dynamic Light Scattering).

FIG. 8A-B depict the Size distribution of unshaved LV (A) and EV (B) batches produced in HEK293-derived cells measured by MADLS (Multiple Angle Dynamic Light Scattering).

FIG. 9A-D depict the GFP expression overtime (up to 14 days) of cell lines transduced with unshaved LVs at MOI 1 to 30. Quantification was performed by flow cytometry. A) Percentage of GFP expressed by HEK293 cells. B) Percentage of GFP expressed by Jurkat cells. C) Mean fluorescence intensity of GFP on transduced HEK293 cells. D) Mean fluorescence intensity of GFP on transduced Jurkat cells.

FIG. 10 summarizes the general workflow for upstream and downstream processing of EVs and LVs produced in HEK293-derived cells.

FIG. 11A-B depict the solubility of cationic polymers in water, 100 mM acetate buffer pH 5 or acetic acid when dissolved at a concentration of 5 mg/ml and analyzed by Dynamic Light Scattering. A) particle count in solution reflects the number of nanoparticles resulting from polymer auto-assembly; B) measured sizes of polymer self-assemblies. The grey area corresponds to the expected sizes of EVs or LVs coated or not with polymers and thus the exclusion zone for polymers that tend to form self-assemblies in solution.

FIG. 12 depicts the size difference measured by Dynamic Light Scattering after Proteinase K (PK) treatment of 3.6×10⁹ EVs particles produced in HEK293-derived cells with increasing concentrations of PK for 10 min at 25° C.

FIG. 13A-C depict the detection by direct ELISA of VSV-G protein on unshaved LV and EV batches produced in HEK293-derived cells. Titration performed with A) anti-VSVG neutralizing monoclonal antibody 8G5F11, B) anti-VSVG monoclonal antibody 1E9F9 and C) anti-VSVG polyclonal antibody.

FIG. 14A-D depict the detection by sandwich ELISA of tetraspanins and VSV-G protein on unshaved LV and EV batches produced in HEK293-derived cells. A) capture with anti-VSVG 8G5F11 and detection with anti-human CD9, B) capture with anti-VSVG 8G5F11 and detection with anti-human CD81, C) capture with anti-VSVG 1E9F9 and detection with anti-human CD9 and D) capture with anti-VSVG 1E9F9 and detection with anti-human CD9.

FIG. 15A-F depict the characterization by sandwich ELISA of shaved versus unshaved LV produced in HEK293-derived cells. Shaving was performed for 15 min with 10, 500, 1000 and 5000 nM Proteinase K before a 25 minutes treatment with 20 mM AEBSF. Data are normalized on the unshaved signal (dotted line and 0 nM PK condition). A) capture with anti-human CD63 and detection with anti-human CD81, B) capture with anti-human CD81 and detection with anti-human CD9, C) capture with anti-VSVG 8G5F11 and detection with anti-human CD9, D) capture with anti-VSVG 1E9F9 and detection with anti-human CD9, E) capture with anti-VSVG 8G5F11 and detection with anti-human CD81 and F) capture with anti-VSVG 1E9F11 and detection with anti-human CD81.

FIG. 16A-B depict the transduction efficiency of shaved and unshaved LV in Jurkat cells. Shaving was performed for 15 min with 10, 500, 1000 and 5000 nM Proteinase K before a 25 minutes proteinase K treatment with 20 mM AEBSF. Shaved LV samples and unshaved LV were used to transduce Jurkat cells at various particles concentration: 1.6e9 particles equivalent to MOI 5 (top panel) and 6.1e9 particles equivalent to MOI 20 (low panel). GFP expression (A) was monitored by flow cytometry from day 3 to day 20 post-transduction with the percentage of GFP expressing cells and the mean fluorescence intensity (MFI) of GFP. Vector copy number (B) was quantified by qPCR on DNA extracted from transduced cells from day 3 to day 20.

FIG. 17A-B depict the transduction efficiency of shaved and unshaved LV in primary activated T cells. Shaving was performed for 15 min with 500 and 5000 nM Proteinase K before a 25 minutes proteinase K treatment with 20 mM AEBSF. Shaved LV samples and unshaved LV were used to transduce activated primary human T cells at various particles concentration: 1.9e8 particles equivalent to MOI 5 (top panel) and 7.7e8 particles equivalent to MOI 20 (low panel). GFP expression was monitored by flow cytometry from day 4 to day 14 post-transduction with the percentage of GFP expressing cells (A) and the mean fluorescence intensity (MFI) of GFP (B). Vector copy number was quantified by qPCR on DNA extracted from transduced cells at MOI 5 on days 4 and 7 (B, top panel, stars).

FIG. 18A-B depict the impact of formulation buffer on Jurkat cells viability and LV transduction. (A) Viable Jurkat cell proportion after 2 hours incubation with 25%, 50%, 75%, 87.5% or 100% of Formulation mediums at pH5 or pH7.4. (B) Percentage of GFP expression after transduction of a VSVG LV with 0, 1, 50 and 75% of formulation buffer. P: Phosphate, C: Citrate, T: Tris, A: Acetate, S: Succinate, H: Histidine, OS: Sucrose & NaCl.

FIG. 19 depicts the size difference measured by Dynamic Light Scattering after Proteinase K (PK) treatment of 5.9×10⁹ LV particles produced in HEK293-derived cells with increasing concentrations of PK for 5 min at 25° C. Proteinase K inhibition with 20 mM AEBSF final was carried out during 25 min.

FIGS. 20 A-D depict the size distribution of unshaved, shaved LV or shaved and filtered LVs produced in HEK293-derived cells measured by MADLS (Multiple Angle Dynamic Light Scattering). Four independent shaving reactions (Batch 1 panel A, batch 2 panel B, batch 3 panel C and batch 4 panel D) were performed on 1.43 x 1011 LV particles with a 15 min incubation step with Proteinase K at 5 UM followed by 25 min inhibition with 20 mM AEBSF. Reaction products were filtered or not through a succession of 0.45 and 0.22 μm PVDF membranes.

FIGS. 21 A-B depict the variation of zeta potential (panel A) and hydrodynamic diameter (z-average as a measure of the size-panel B) as a function of the pH for unshaved or shaved and filtered LVs particles produced in HEK293-derived cells. Viral particles were diluted 10-fold in DPBS-Sucrose and pH was adjusted before measurement by MADLS (Multiple Angle Dynamic Light Scattering).

FIGS. 22 A-B depict the variation of hydrodynamic diameter (z-average as a measure of the size) and zeta potential of polymeric nanoparticles as a function of the viral particle concentration (panel A) or cationic polymers concentration (panel B) for shaved and filtered LVs particles produced in HEK293-derived cells. Viral particles and cationic polymers were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering).

FIG. 23A-B depict the variation of hydrodynamic diameter (z-average as a measure of the size) (panel A) and size distribution (Pdl for Index of Polydispersity) (panel B) of polymeric nanoparticles obtained from two different batches of shaved (with 5000 nM Proteinase K) and filtered LVs particles produced in HEK293-derived cells. Viral particles and cationic polymers were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering).

FIG. 24A-B depict the size distribution (panel A) and variation of zeta potential (panel B) of polymeric nanoparticles for shaved and filtered LVs particles produced in HEK293-derived cells. Viral particles, H-PBAE (cationic polymer) and HA (anionic polymer) polymers were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering). Nanoparticles obtained after first coating step were added to anionic HA polymers and coating reaction was performed 30 min at 25° C. before measurement by MADLS.

FIG. 25A-D depict the variation of hydrodynamic diameter (z-average as a measure of the size) (panels A & C) and size distribution (panels B & D) of polymeric nanoparticles for shaved and filtered LVs particles produced in HEK293-derived cells. Viral particles, cationic (PBAE-447. H-PBAE and R-PBAE) and anionic polymers (Heparosan and HA) were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering). Nanoparticles obtained after first coating step were added to Heparosan or HA anionic polymers and coating reaction was performed 30 min at 25° C. before measurement by MADLS.

FIG. 26A-B depict the detection by sandwich ELISA of CD19-CAR and tetraspanin proteins on unshaved LV coding for CD19-CAR or GFP (batch 1) produced in HEK293-derived cells. A) capture with anti-human CD63 or CD81 and detection with CD19 recombinant protein or anti-FMC63 idiotype antibody, B) capture with recombinant CD19 protein and detection with anti-human CD81 or CD9 antibody.

### DETAILED DESCRIPTION OF THE INVENTION

The invention encompasses EVs and methods for making, modifying, and using them. Before the present compositions, methods, and other embodiments are disclosed and described, it is to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

As used herein, the term “in vitro” refers to events that occur in an artificial environment, e.g., in a test tube or reaction vessel or in cell culture, etc., rather than within an organism.

As used herein, the term “in vivo” refers to events that occur within an organism.

As used herein, the term “ex vivo” refers to events that occur outside of an organism with material taken directly from an organism.

As used herein, the term “contact” or “contacting” refers to the placing of two compounds or compositions (e.g., cells and extracellular vesicles) together with no physical barriers between them (e.g., in the same tube in solution).

As used herein, the term “purified” refers to a PNP (polymeric nanoparticle), EV, LV, capsid, or other composition or molecule (e.g., protein) that has been separated from some other constituent with which it is associated in a mixture. For example, “purified” EVs can be those separated from the cells that produce them or from proteases with which they have been treated. Likewise, “purified” capsids can be those separated from a cell lysate or from the outer lipidic membrane components of a retroviral or lentiviral particle. The degree of purification based on the extent of removal of other components. Thus, purity can range from 50% purity or less to 75%, 80%, 90%, 95%, 96%, 97%, 98%, 99%, or more. For example, a 50% purified EV can have 50% of the protease with which it has been treated removed.

### Extracellular Vesicles (EVs)

In various embodiments, the invention encompasses EVs and methods for making and using them.

The invention encompasses 1, 10, 10², 10³, 10⁴, 10⁵, 10⁶, 10⁷, 10⁸, 10⁹, 10¹⁰, 10¹¹, 10¹², 10¹³, 10¹⁴, 10¹⁵, etc. EVs or PNPs, or any range between any of these amounts (e.g. from 10⁵ to 10⁸ EVs or PNPs).

The invention encompasses concentrations of EVs or PNPs of 10²/ml, 10³/ml, 10⁴/ml, 10⁵/ml, 10⁶/ml, 10⁷/ml, 10⁸/ml, 10⁹/ml, 10¹⁰/ml, 10¹¹/ml, 10¹²/ml, 10¹³/ml, 10¹⁴/ml, 10¹⁵/ml, etc. or any range between any of these amounts (e.g. from 10⁹/ml to 10¹²/ml).

In some embodiments, the EVs are endosome-derived (“exosomes”) with a diameter of ~30 nm to ~150 nm. Preferably, the EVs are generated by membrane outward budding (“ectosomes” or “microvesicles”) with a diameter of ~50 nm to ~1000 nm.

In some embodiments, the EVs are ectosomes of ~100 nm to ~300 nm in size. In one embodiment, the EVs are ectosomes of ~150 nm in size.

Preferably, the EVs batches contain less than 600 ng/ml of residual DNA and less than 15 μg/mL of host cell proteins.

The EVs can be generated from extracellular most types of cells. Preferably, the EVs are generated from eukaryotic cells. Eukaryotic cells include yeast, avian, and mammalian cells, such as chicken, mouse, primate, monkey, human, hamster, rat, etc.

In some embodiments, the eukaryotic cells are JURKAT, VERO, HELA, COS-7, CHO, HEK293, BHK-21, 293T, MDCK, BSC-1, NSO, PER.C6, VERO, CRL-7030, HS578BST, NIH 3T3, HEPG2, A549, DAUDI, R1.1, BW-5147, L-M, BSC-40, YB/20, P3X63Ag8.653, SP2/0-Ag14, or BMT10 cell lines. Many cell lines are readily available from, for example, American Type Culture Collection (ATCC).

In some embodiments, the eukaryotic cells are from an animal, such as avian or mammalian sources, such as chicken, mouse, primate, monkey, human, hamster, rat, etc.

In some embodiments, the eukaryotic cells are fibroblasts, immune cells, or stem cells, including iPSCs.

In some embodiments, the eukaryotic cells express viral proteins, such as Vesicular Stomatitis Virus (VSV), retrovirus, and measles viral proteins. These proteins include viral envelope proteins, particularly ones that are being used to pseudotype a vector. These include the envelope proteins of murine leukemia virus (MULV), the gibbon ape leukemia virus (GALV), the feline endogenous RD114 retrovirus, Moloney MULV 4070A, Moloney MULV strain 10A1, the envelope glycoprotein of vesicular stomatitis virus (the VSV-G protein), the rabies virus glycoprotein, and the measles virus hemagglutinin and fusion glycoproteins.

In some embodiments, the eukaryotic cells express retroviral proteins. Retroviral proteins include Gag, Pol, Rev, and Env proteins. Retroviral proteins further include a capsid, matrix, nucleocapsid, protease, reverse transcriptase, and integrase proteins. Retroviral proteins also include additional retroviral accessory proteins, such as Tat, Nef, Vif, Vpr, Vpu, and Vpx.

One or more of the retroviral proteins can be transiently or stably expressed by the cells. Preferably, the retroviral Gag and Pol proteins are expressed by transient transfection of the cells. Preferably, RNAs comprising retroviral packaging sequences are expressed by transient transfection of the cells.

In some embodiments, the EVs comprise a retroviral envelope (e.g., lentiviral such as an HIV-1 Env) protein on their outer surface. In some embodiments, the EVs comprise a CAR on their outer surface.

In some embodiments, the retrovirus is an avian leukosis virus, Rous sarcoma virus (RSV), mouse mammary tumor virus (MMTV), murine leukemia virus (MuLV), feline leukemia virus (FLV), bovine leukemia virus (BLV), or human T-lymphotropic virus (HTLV).

In some embodiments, the retrovirus is a lentivirus (LV), such as human immunodeficiency virus type 1 (HIV-1), human immunodeficiency virus type 2 (HIV-2), simian immunodeficiency virus (SIV), or feline immunodeficiency virus (FIV).

In some embodiments, the EVs of the invention comprise a payload for delivery to another cell. This payload can include proteins, nucleic acids, or small molecules. The protein or nucleic acid can be expressed in or introduced into the cell from which the EV is generated. In this way, the payload is associated with the EV upon generation from the cell. Alternatively, the EVs can be loaded with the payload after purification. Payloads include one or more therapeutic proteins and nucleic acids encoding one or more therapeutic proteins.

In some embodiments, the therapeutic proteins are cytokines or growth factors, such as interferons, interleukins, lymphokines, and tumor necrosis factors, for example, IL-1, IL-2, IL-4, IL-6, IL-10, IL-11, IL-13, IL-17, IL-35, TNFα, IFN-α, IFN-β, IFN-γ, TGF-β, GM-CSF, M-CSF, G-CSF, EPO, BMP, EGF, FGF, and VEGF.

In some embodiments, the therapeutic proteins are antibodies or other inactivating molecules that target Programmed Death 1 (PD-1), Cytotoxic T-Lymphocyte Antigen 4 (CTLA-4), LAG3, TIM3, BTLA, BY55, TIGIT, LAIR1, SIGLEC10, 2B4, PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3.

Such therapeutic proteins include chimeric antigen receptors and chimeric costimulatory receptors (CCRs).

Chimeric antigen receptors (CARs) include those directed against carbonic anhydrase IX, (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD117, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (ACHR), folate receptor-a, FLT3, Ganglioside G2 (GD2), Ganglioside G3 (GD3), human Epidermal Growth Factor Receptor 2 (HER-2), human telomerase reverse transcriptase (HTERT), Interleukin-13 receptor subunit alpha-2 (IL-13Ra2), K-light chain, kinase insert domain receptor (KDR), Lewis Y (LeY), L1 cell adhesion molecule (LICAM), melanoma antigen family A1 (MAGE-A1), Mucin 16 (MUC16), Mucin 1 (MUC1), Mesothelin (MSLN), ERBB2, MAGE-A3, MAGE-A4, p53, MART1, GP100, Proteinase3 (PR1), Tyrosinase, Survivin, hTERT, EphA2, NKG2D ligands, cancer testis antigen NY-ESO-1, oncofetal antigen (h5T4), prostate stem cell antigen (PSCA), prostate-specific membrane antigen (PSMA), ROR1, tumor associated glycoprotein 72 (TAG-72), vascular endothelial growth factor R2 (VEGF-R2), and Wilms tumor protein (WT-1), BCMA, NKCS1, EGFIR, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, B-cell activating factor receptor (BAFF-R), Thymic stromal lymphopoietin (TSLPR), SLAM Family Member 7 (SLAMF7) and G-protein coupled receptor family C group 5 member D (GPRC5D).

Chimeric antigen receptors (CARs) include those directed against CD19, CD20, CD22, CD30, CD33, CD38, CD123, CD133, CD147, CD317, FLT3, BCMA, DR4, CS-1, CLL-1, ADGRE2, HER2, HSP70, VEGF, GD2, GD3, MUC1, MUC16, NKR2, NKG2D ligands, CYP1B1, SP17, PRAME, and WT1. Chimeric antigen receptors (CARs) are preferably those directed against CD19, present in B-cell-derived cancers such as acute lymphoblastic leukemia (ALL) and diffuse large B-cell lymphoma (DLBCL); CD30, present in refractory Hodgkin's lymphoma; CD33, CD123, ADGRE2 and FLT3, present in acute myeloid leukemia (AML); and BCMA, present in multiple myeloma.

Preferably, the CAR contains an extracellular antigen binding domain, a transmembrane domain, a hinge domain, a costimulatory signaling region, and an intracellular domain. Chimeric antigen receptors (CARs) include first generation, second generation, and third generation CARs. See US 2020/0317777, which is hereby incorporated by reference.

First generation CARs are typically composed of an extracellular antigen-binding domain (e.g., a scFv), which is fused to a transmembrane domain, which is fused to cytoplasmic/intracellular signaling domain. Second generation CARs add intracellular signaling domains from various co-stimulatory molecules (e.g., CD28, 4-1BB, ICOS, OX40, CD27, CD40/My88, and NKG2D) to the cytoplasmic tail of the CAR to provide additional signals to the T cell. Third generation CARs comprise those that provide multiple co-stimulation (e.g., CD28 and 4-1BB) and activation (e.g., CD3z) domains.

In some embodiments, the extracellular antigen binding domain of the CAR comprises a human scFv, humanized scFv, murine scFv, F (ab) 2, VHH (Camelid Heavy chain), or a partial natural ligand for a cell surface receptor.

In some embodiments, the transmembrane domain of the CAR comprises a native or modified CD8, CD28, CD3z, CD40, 4-1BB, OX40, CD84, CD166, CD8a, CD8b, ICOS, ICAM-1, CTLA-4, CD27, CD40/My88, NKG2D, synthetic polypeptide or combination thereof.

In some embodiments, the CAR contains a hinge region. In some embodiments, the hinge domain of the CAR comprises a CD8 polypeptide, a CD28 polypeptide, a CD3z polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKG2D peptide, a synthetic polypeptide (not based on a protein associated with the immune response), or a combination thereof. The hinge spacer region can be the hinge region from IgG1, or the CH2 and CH3 region of immunoglobulin and portions of CD3, a portion of a CD28 polypeptide.

In some embodiments, the CAR contains one or more costimulatory signaling regions. In some embodiments, the costimulatory signaling region comprises at least one native or modified CD8, CD3z, CD27, CD28, CD40, 4-1BB, OX40, CD84, CD166, CD8a, CD8b, ICOS, ICAM-1, CTLA-4, CD40/My88, NKG2D, synthetic polypeptide, or combinations thereof. Costimulatory ligands include CD80, CD86, CD70, OX40L and 4-1BB ligands.

In some embodiments, the intracellular domain of the CAR comprises a CD3z polypeptide modified or not on ITAM motifs.

In some embodiments, the therapeutic proteins are combinations of different types of therapeutic proteins.

In some embodiments, the payload is a gene editing system, such as a TALEN, ZFN or CRISPR-based gene editing system.

### Production Methods

The invention encompasses methods for the production of EVs. In some embodiments, the EVs are prepared by separating the EVs from the cells producing them (“production cells”) by well-known techniques in the art. If the production cells are adherent, the supernatant can be collected from the cells or the cells can be removed from the plate or flask (e.g., with trypsin), and the production cells can be removed by centrifugation or filtration.

If the production cells are non-adherent, the production cells can be removed by centrifugation or filtration. The supernatant can be subjected to centrifugation and/or filtration to remove whole cells and/or other contaminants.

To increase EVs production, specialized cell culture methods, including specially made culture flasks or 3D culture, can be used. See Du et al., Pharmaceutics 2022, 14:2236, which is hereby incorporated by reference. Ca2+-dependent regulation induction, stressed culture conditions stimulation (hypoxia, low pH, electrical stimulation, liposome stimulation, acoustic treatments, and drug stimulation), and specialized EV separation methods (tangential flow filtration) can also be used to increase production.

EVs can be purified by numerous well-known techniques in the art. See Chen et al., Front. Bioeng. Biotechnol. 2022, 9:811971, 1-18, which is hereby incorporated by reference. Isolation techniques include ultrafiltration, multimodal chromatography, immunoaffinity, ultracentrifugation, acoustic devices, and microfluidic devices.

For example, these techniques can be used to remove proteins, lipids and nucleic acids, and other molecules and structures smaller than 50, 100, or 150 nm. Similarly, these techniques can be used to remove proteins, lipids and nucleic acids, and other molecules and structures larger than 200, 300, 400, or 500 nm.

In some embodiments, the technique removes at least 50%, 60%, 70%, 80%, 85%, 90%, 95%, 97% or 99% of the molecules and structures smaller than 150 nm.

In some embodiments, the technique removes at least 50%, 60%, 70%, 80%, 85%, 90%, 95%, 97% or 99% of the molecules and structures larger than 300 nm.

### Extracellular Vesicle Shaving

The invention encompasses methods of modifying the surface of EVs. The EVs can be treated with enzymes to remove moieties on their outer surface (“shaving”). For example, the EVs can be treated with a protease to cleave proteins that are accessible on the surface of EVs.

The invention encompasses a method of modifying the surface of EVs comprising a) treating EVs with a protease to remove, totally or partially, proteins on the outer surface of the EVs; b) inactivating the protease and c) purifying the treated EVs from the protease. In some embodiments, the EVs are treated with the protease in the presence of the EV producer cells. The producer cells can be removed by centrifugation and/or filtration.

The invention encompasses a method of modifying the surface of purified EVs comprising a) purifying EVs from a cell; b) treating the EVs with a protease to remove proteins on the outer surface of the EVs; c) inactivating the protease; and d) purifying the treated EVs from the protease and protein debris.

Preferably, the EVs comprise a retroviral capsid. The retroviral capsid can contain the retroviral proteins necessary for reverse transcription, with or without the ability for integration. More preferably, the capsid comprises RNA molecules encoding a protein, preferably a therapeutic protein (e.g. a CAR or CCR). In some embodiments, the capsid comprises RNA molecules encoding a gene editing system, such as a TALEN, ZFN or CRISPR-based gene editing system.

The method can further comprise coating the EVs with one or more polymers to generate a polymeric nanoparticle (PNPs). Preferably, the polymeric nanoparticle comprises a targeting moiety, most preferably for T cells (e.g., a CD3 targeting agent, such as, an anti-CD3 antibody or aptamer) or natural killer (NK) cells (e.g., a CD56 or NKG2D targeting agent, such as, an anti-CD56 or NKG2D antibody or aptamer). See Menon et al., Cancers 2023, 15:1189 and Lian et al., Int. J. Mol. Sci. 2022, 23, 164 which are hereby incorporated by reference.

The method can further comprise contacting the coated EVs/PNPs, with or without a targeting agent, with target cells. In one embodiment, the target cells are T cells. In some embodiments, the target cells are NK cells or hematopoietic stem cells. The coated EVs/PNPs and cells can be contacted in vitro, ex vivo, or in vivo.

The protease can be a mixture of proteases or a purified protease. In one embodiment, the protease is an endopeptidase. In one embodiment, the protease is an exopeptidase. In some embodiments, the protease is from the class of Serine, Aspartic, Metallo, or Cysteine proteases.

Proteases include endopeptidases, such as, Proteinase K, Trypsin, Chymotrypsin, Endoproteinase Asp-N, Endoproteinase Arg-C, Endoproteinase Glu-C, Endoproteinase Lys-C, Pepsin, Thermolysin, Elastase, Papain, Subtilisin, and Clostripain.

Proteases include endopeptidases, such as, Carboxypeptidase A, Carboxypeptidase B, Carboxypeptidase P, Carboxypeptidase Y, Cathepsin C, Acylamino-acid releasing enzyme and Pyroglutamate Aminopeptidase.

Proteases can be chosen based on the proteins which are desired to be cleaved or left uncleaved. For example, VSV-pseudotyped lentiviral vectors are less susceptible to Trypsin inactivation than other envelope-pseudotyped lentiviral vectors. See Dautzenberg et al., Gene Therapy 2021, 28:89-104, which is hereby incorporated by reference.

The protease is mixed with the EVs, preferably after isolation, and incubated for a period of time at an appropriate temperature and concentration to allow cleavage of the protein(s) of interest.

The extracellular parts of vectors surface proteins can be digested by proteinase K or other broad-spectrum proteases on purified or non-purified EVs.

In one embodiment, the protease is proteinase K to cleave peptide bonds adjacent to the carboxyl group of aliphatic and aromatic amino acids. The enzymatic treatment can be performed by direct addition of recombinant proteinase K (e.g., Invitrogen, 25530015) into vectors samples at a final concentration of about 0.01, 0.02, 0.03, 0.04, 0.05, 0.06, 0.07, 0.08, 0.1, 0.15, 0.2, 0.25, 0.3, 0.4, 0.5, 1, 2, 5, 10, 20, 50, 100, 200, or 500 μg/ml, or any range between any of these amounts (e.g. from 0.03-0.3 μg/ml) for 10⁶, 10⁷, 10⁸, 10⁹, 10¹⁰ or 10¹¹ EVs for at least 1, 2, 5, 10, 15, 30, 60 minutes to 2, 4, 6, 12, or 24 hours or any range between any of these amounts (e.g. from 60 to 180 min) at 20, 25, 30, 35, 37, or 40° C. at a pH of 6.5 to 9.5 with or without agitation. In one embodiment, 3.6×10⁹ particles EVs are treated with 0.03-10 μg/ml of proteinase K for 10 min at 25° C.

After incubation, the proteolysis can be stopped with 10 mM phenylmethylsulfonyl fluoride (PMSF, Thermo Scientific, 36978), 4-(2-Aminoethyl)benzenesulfonyl fluoride hydrochloride (AEBSF, Thermo Scientific, 78431) or diisopropylfluorophosphate (DFP). The excess of PMSF, AEBSF or DFP ensures a complete inactivation of the enzyme before proceeding to purification and encapsulation.

Preferably, the protease removes cellular and/or viral proteins on the outer surface of the EV. Preferably, the protease removes all or part of one or more of the following cellular proteins: MHC, CD24, CD29, CD44, CD55, CD146, CD326, Cadherin, ZO-1, Occludin, Tetraspanins (CD9, CD63, CD81, CD82), EGFR, or integrins.

Preferably, the protease removes all or part of one or more of the following viral proteins: retroviral/lentiviral envelope protein, measles virus envelope protein, or Vesicular Stomatitis Virus (VSV) envelope protein.

Preferably, the protease removes all or part of one or more of the extracellular therapeutics proteins, for example, a chimeric antigen receptor (CAR) or a chimeric costimulatory receptor (CCR).

In some embodiments, the protease removes enough cellular and/or viral protein on the outer surface of the EV to lose LV functionality. In some embodiments, the protease removes at least 10%, 20%, 30%, 40%, 50%, 75%, 80%, 90%, 95%, 96%, 97%, 98%, 99%, or 100% of the detectable cellular and/or viral protein on the outer surface of the EV.

In some embodiments, the protease removes at least 50%, 75%, 80%, 90%, 95%, 96%, 97%, 98%, 99%, or 100% of the detectable CD9, CD24, CD29 (Integrin Beta 1), CD44, CD63, CD81, CD82, CD146, CD326 (EPCAM), VSV protein, extracellular parts of CAR and CCR on the outer surface of the EV.

The removal of cellular, viral and therapeutic proteins from the outer surface of the EV can be measured by measuring the levels of these proteins prior to and after protease treatment, such as by the techniques set out in the examples herein, such as by measuring the binding of antibodies to these proteins.

In some embodiments, the protease removes at least 5%, 10, 20%, 30% 40% 50%, 75%, 80%, 90%, 95%, 96%, 97%, 98%, 99%, or 100% of the detectable chimeric antigen and/or costimulatory receptor on the outer surface of the EV.

In some embodiments, fragments of the cellular and/or viral protein and/or therapeutic proteins remain in the membrane or interior of the EV. In some embodiments, fragments of the Vesicular Stomatitis Virus (VSV) envelope protein remain in the membrane or interior of the EV. In some embodiments, fragment and/or domains of the chimeric antigen receptor and chimeric costimulatory receptor remain in the membrane or interior of the EV.

In some embodiments, a protease inhibitor is added after the protease treatment to inactivate the protease. Protease inhibitors include a Protease Inhibitor Cocktail or individual protease inhibitors. Preferred protease inhibitors are Phenylmethanesulfonyl fluoride (PMSF, Thermo Scientific, 36978), 4-(2-Aminoethyl)Benzenesulfonyl Fluoride Hydrochloride (AEBSF, Thermo Scientific, 78431), and Diisopropylfluorophosphate (DFP).

The invention encompasses methods for removing protease from the treated EVs. Removal methods include tangential flow filtration (TFF) and other filtration methods, such as size-exclusion. In some embodiments, one of the following is used: EXOEASY Maxi kit (Qiagen, 76064), EXOQUICK-TC ULTRA (System Biosciences, EQULTRA-20TC-1), AMICON Ultra-15 100 kDa (Merck, UFC910008) or MACROSEP Advance 100K (Pall, MAP100C36).

### Purification Post-Shaving

The removal of impurities generated during the proteolysis treatment is essential before polymer coating. The purification strategies used are based on the removal of impurities from the shaved vector using molecular weight/size-based methods.

For volumes of shaved vectors below 10 mL, the ultrafiltration method using Polyvinylidene fluoride (PVDF), Polyethersulfone (PES), modified Polyethersulfone (mPES) or cellulose-centrifugal filter units (Millipore, AMICON ultra filter) of 100 to 500 kD molecular weight cut-off (MWCO) is used. The vector concentration step is followed by a diafiltration with at least 3 steps of 1/10e dilution for proper buffer exchange into the final polymer-coating buffer.

For larger volumes and increased purity, size exclusion chromatography (SEC), multimodal chromatography followed by a tangential flow filtration (TFF, hollow fiber or cassette) or a TFF step alone is used to purify, concentrate and formulate in the specific polymer-coating buffer.

### Evaluation of the Shaving Process

### Multiplex Detection of Surface Proteins

The MACSPLEX Exosome kit (Miltenyi Biotec, 130-108-813) can be used to compare surface epitopes detected on EVs before and after shaving. The method allows the quantification of the expression of up to 37 human membrane epitopes using specific antibodies. The overnight protocol is followed according to manufacturer's instructions using 10⁷, 10⁸ or 10⁹ EV particles and detection with anti-CD9, or anti-CD63, or anti-CD81 or all three antibodies simultaneously.

### Direct and Sandwich ELISA

EVs produced in HEK293T or derived should express human tetraspanins CD9, CD63 and CD81 at their surface and CD24, CD29, CD44, CD146 and CD346 membranes proteins. In addition, lentiviral vectors may express VSV-G proteins as well as transmembrane transgenes such as CAR and CCR. Thus, shaving efficiency can be measured by the lack or decrease of binding of anti-CD9 (Biolegend, 312102, clone HI9a; Invitrogen, MA5-33125, clone 3A2), CD24 (Biolegend, 311102, clone ML5), CD29/Integrin beta 1 (Invitrogen, MA5-17103, clone 3B6), CD44 (Invitrogen, MA5-15462, clone 8E2F3), CD63 (Invitrogen, MA5-30187, clone 142; Biolegend, 353039, clone H5C6, CD81 (Biolegend, 349502, clone 5A6; Invitrogen, MA5-33123, clone 9F7), CD146 (Invitrogen, MA5-29414, clone 12), CD326 (Invitrogen, 14-9326-82, clone 1B7), VSVG (Sigma-Aldrich, MABF2337-100UG, clone 8G5F11; Sigma-Aldrich, MABF2321-100UL, 1E9F9), CAR (for FMC63 scFv: Miltenyi Biotec, 130-127-983, clone REA1297; Miltenyi Biotec, 130-127-984, clone REA1298) or CCR antibodies to the surface of EVs, or antigen like CD19 (Miltenyi Biotec, 130-129-550; ACROBiosystem, CD9-H52H2) to CAR and CCR, either by direct ELISA or sandwich ELISA.

For direct ELISA, 108 EVs per well can be coated in a MAXISORP 96-well plate (Thermo Scientific, NUNC, 439454) overnight in DPBS 1X (Gibco, 14190094). After 2 hours saturation in 2% BSA (Thermo Scientific, 37525), a dilution range of primary antibody or antigen (listed above) is incubated for 1.5 hours. After washing with a solution of DPBS 1X and 0.05% Tween (Sigma, P9416), secondary antibodies or streptavidin/avidin coupled to HRP are incubated for 30 min. The HRP coupled reagents used are: goat anti-mouse IgG (H+L) HRP conjugate (Invitrogen, 31430) at 1/5 000, goat anti-mouse IgG (H+L) HRP conjugate (Invitrogen, 31460) at 1/10 000, Streptavidin-HRP (Thermo Scientific, 21130) at 1/10 000 or Avidin-HRP (Biolegend, 405103) at 1/5 000. After well washes, 1X TMB substrate solution (Invitrogen, 00-4201-56) is added for 10 min and stopped with a stop solution (Thermofisher, N600). Absorbance is measured using a GIOMAX Discover (Promega, GM3000) at 450 nm.

Antibody pairs have been selected for simultaneous detection of two different protein surface epitopes (tetraspanins, VSVG and/or CAR). Sandwich ELISA is performed with an overnight coating of a primary antibody, a saturation step and then an incubation of a range of EV for 2 hours at room temperature. A biotinylated antibody is incubated for 1.5 hours, followed by washing and incubation of streptavidin-HRP (Thermo Scientific, Pierce, 21130) at 1/10 000e dilution for 30 min. Absorbance is measured as described for direct ELISA.

### Western Blot for Cytoplasmic Domains Detection

Tetraspanins, VSVG, CAR and other proteins expressed at the surface of EVs are transmembrane proteins. Even if the surface domains are removed, their cytoplasmic domains are intact and not altered by the shaving procedure. Thus, EVs integrity after shaving can be assessed by western blot using specific antibodies targeting the cytoplasmic domains of the proteins CD9 (Invitrogen, MA5-33125, clone 3A2), CD63, CD81 (AbboMax, 630-910), CD326 (Abcam, ab233919, clone EGP40-1110), VSVG (Abcam, ab50549, clone P5D4), CD3 zeta (Sigma-Aldrich, MABF3032, clone 6B10.2) and CD28 (Invitrogen, MA5-36070, clone RM404; Invitrogen, PA5-116751). The EVs samples are first lysed in RIPA buffer (Thermo Scientific, 89900) or equivalent lysis buffer containing NP-40 or Triton to extract proteins from EV samples. Extracted and denatured EVs samples are loaded on NUPAGE 4-12% Bis-Tris gels (Thermofisher, NP0335BOX) and migration is performed in MES SDS running buffer (Thermofisher, NP0002) according to manufacturer's instructions. Then, the transfer is performed using the IBLOT 2 dry blotting system (Thermofisher, IB21001) on nitrate cellulose membrane (Invitrogen, IB23002). The blot is blocked in a solution of PBS 1X, 0.1% Tween 20 (PBST) and 3% BSA for 1 hour and washed in PBST three times. Then, primary antibodies are incubated in PBST with 0.2% BSA according to manufacturer's instructions, followed by three washes and incubation of HRP-conjugate reagent. Revelation is performed using the SUPERSIGNAL WEST PICO plus chemiluminescent substrate (Thermofisher, 34580) before acquisition on an IBRIGHT FL1500 Imaging system (Thermofisher, A44241).

### Transduction Efficiency

Titration of shaved retroviral (e.g., lentiviral) vectors can be performed by flow cytometry or by qPCR, to confirm the loss of infectivity after shaving on two different cell lines: HEK293T (ATCC, CRL-3216) and Jurkat (ATCC, TIB-152) cells.

### Flow Cytometry

Four hours before transduction, HEK293T cells are seeded in 48-well plates (Corning, 3548) at 8×10⁵ cells/well in DMEM (Gibco, 61965026) containing 10% FBS (Gibco, 10091148), 1% Penicillin/Streptomycin (PS) (Gibco, 15140122) and incubated at 37° C. and 5% CO₂. Just before transduction, Jurkat cells are seeded at 8×10⁵ cells/well in RPMI (Gibco, A1049101) containing 10% FBS, 1% PS. A serial dilution of unshaved and shaved vectors (at a same physical titer) is incubated on cells at 37° C., 5% CO₂. After 2 hours, fresh culture medium is added into each well and incubation is extended to 3 days. At day 3, cells are collected (with trypsinization step for HEK293T using TrypLE, Gibco, 12563011), washed in DPBS 1X, stained with Zombie NIR (Biolegend, 423106) diluted in DPBS 1X at 1/1000 for live/dead discrimination and then fixed with fixation buffer (Biolegend, 420801) for GFP encoding vectors (SEQ ID NO: 3) discrimination. For CD19-CAR encoding vectors (SEQ ID NO: 4), additional staining steps before fixation are performed by incubating first the CD19 CAR detection reagent (Miltenyi Biotec, 130-129-550) for 10 min, washed and then followed by incubation with biotin antibody-VB515 (Miltenyi Biotec, 130-110-957, clone REA746).

The number of viable fluorescent-positive cells after transduction with shaved or non-shaved vectors is determined using a flow cytometer (ATTUNE NXT, Thermofisher) and the infectious titer is quantified using cells expressing between 5 and 25% of GFP or CD19 CAR.

qPCR Reactions for Infectious Titer Determination

Four hours before transduction, HEK293T cells are seeded in 24-well plates (Corning, 3513) at 8×10⁴ cells/well in DMEM (Gibco, 61965026) containing 10% FBS (Gibco, 10091148), 1% Penicillin/Streptomycin (PS) (Gibco, 15140122) and incubated at 37° C. and 5% CO₂. Just before transduction, Jurkat cells are seeded at 8×10⁴ cells/well in RPMI (Gibco, A1049101) containing 10% FBS, 1% PS. Cells are transduced with a minimum of 3 successive dilutions of shaved or unshaved EVs and cultures for 72 h at 37° C. and 5% CO₂. After 72 hours, cells are harvested and pellets collected. Total genomic DNA from transduced cell-pellets is extracted using the MAXWELL RSC Blood DNA kit (Promega, AS1400) according to manufacturer's instructions. Proviral DNA quantification is performed by qPCR using PERFECTA MULTIPLEX TOUGHMIX (Quantabio, 95147-250). The amplification of integrated sequences is performed using probe and primers specific to the LTR (long terminal repeat sequences).

### Physical Titer

Particle concentration measurement of shaved retroviral (e.g., lentiviral) vectors can be performed as follows to confirm that shaving treatment does not impact the integrity of the vectors produced in HEK293T or derived. Three orthogonal methods are used based on the particles number (NTA), the quantification of capsid P24 protein or the quantification of viral RNA copy (RT-qPCR).

The particle concentration of the vectors produced in HEK293T or derived can be determined by Nanoparticle Tracking analysis using a ZETAVIEW instrument (Particle Metrix GmbH) or by interferometry using the VIDEODROP instrument (Myriade). Before measurement, freshly prepared or frozen-thawed vectors produced in HEK293T or derived can be diluted 1000-fold in DPBS-50 mM Sucrose and homogenized by gentle vortexing.

The detection and quantification of p24 capsid lentiviral vector protein can be performed with the HIV-1 p24 antigen ELISA 2.0 kit (Zeptometrix, 0801002) according to manufacturer's instructions.

The quantification of proviral RNA copies can be performed with the LENTI-X QRT-PCR TITRATION kit (Takara, 631235) according to manufacturer's instructions after the isolation and purification of viral RNA with the MAXWELL RSC TNA viral kit (Promega, AS1330).

### Membrane Integrity

Multi-Angle Dynamic Light Scattering (MALS) can be used to compare global charge (zeta potential), size (average hydrodynamic diameter) and size distribution (Polydispersity index) of the vectors produced in HEK293T or derived before and after shaving and thereby confirm membrane integrity after treatment. Freshly prepared or frozen-thawed EVs can be diluted 16-(zeta potential and Polydispersity index) or 20-fold (average hydrodynamic diameter) in DPBS-50 mM Sucrose and homogenized by gentle vortexing before analysis on a ZETASIZER ULTRA instrument (Malvern Instruments).

### Shaved Extracellular Vesicles (Shaved EVs)

The invention encompasses EVs that have been treated with enzymes to remove moieties on their outer surface (“shaved extracellular vesicles” or “shaved EVs”).

The invention encompasses 1, 10, 10², 10³, 10⁴, 10⁵, 10⁶, 10⁷, 10⁸, 10⁹, 10¹⁰, 10¹¹, 10¹², 10¹³, 10¹⁴, 10¹⁵, etc. shaved EVs, or any range between any of these amounts (e.g. from 105 to 108 EVs).

The invention encompasses concentrations of shaved EVs of 10²/ml, 10³/ml, 10⁴/ml, 10⁵/ml, 10⁶/ml, 10⁷/ml, 10⁸/ml, 10⁹/ml, 10¹⁰/ml, 10¹¹/ml, 10¹²/ml, 10¹³/ml, 10¹⁴/ml, 10¹⁵/ml, etc. or any range between any of these amounts (e.g. from 10⁹/ml to 10¹²/ml).

In some embodiments, the shaved EVs are endosome-derived (“exosomes”) with a diameter of ~30 nm to ~150 nm. Preferably, the shaved EVs are generated by membrane outward budding (“ectosomes”) with a diameter of ~50 nm to ~300 nm.

In some embodiments, the shaved EVs contain less than 50%, 40%, 30%, 20%, 10%, 5%, 4%, 3%, 2%, or 1% of the original level of the detectable cellular and/or viral protein on the outer surface of the EV prior to shaving.

In some embodiments, the shaved EVs contain less than 50%, 40%, 30%, 20%, 10%, 5%, 4%, 3%, 2%, or 1% of the original level of detectable CD9, CD29, CD63, CD81, CD82, CD146, or VSV protein on the outer surface of the EV prior to shaving.

The removal of cellular or viral proteins from the outer surface of the EV can be measured by measuring the levels of these proteins prior to and after protease treatment, such as by the techniques set out in the examples herein.

In some embodiments, the shaved EVs contain less than 50%, 40%, 30%, 20%, 10%, 5%, 4%, 3%, 2%, or 1% of the original level of detectable chimeric antigen receptor on the outer surface of the EV prior to shaving.

Preferably, the shaved EVs have been further purified to remove the protease used to shave them. Removal methods include tangential flow filtration (TFF) and other filtration methods, such size-exclusion or multimodal chromatography.

### Retroviral Capsids

The invention encompasses EVs, preferably shaved EVs, comprising retroviral capsids. The retroviral capsids can be from any retrovirus. Preferably, the retroviral capsids are lentiviral capsids.

The retroviral capsids can be introduced into the EVs by expressing retroviral proteins in the producer cells, by transfection or infection of cells or using a stably-transformed cell line. The cells can express various retroviral proteins from RNA or DNA encoding the retroviral proteins, for example, as exemplified in Dautzenberg et al., Gene Therapy 2021, 28:89-104, which is hereby incorporated by reference.

EVs containing retroviral capsids can be produced by well-known methods, for example, using a three-to four-plasmid production approach as described in Zufferey et al., Nature biotechnol 1997, 15:871-875.

The invention encompasses a method of comprising a) producing a capsid in a cell by expressing retroviral Gag proteins, b) purifying EVs containing the capsid from a cell; c) treating the EVs with a protease to remove proteins on the outer surface of the EVs; and d) purifying the treated EVs from the protease.

The method can further comprise coating the EVs with one or more polymers to generate a polymeric nanoparticle. Preferably, the polymeric nanoparticle comprises a targeting moiety, most preferably for T cells (e.g., an CD3 targeting agent, such as, an anti-CD3 antibody or aptamer) or natural killer (NK) cells (e.g., a CD56 or NKG2D targeting agent, such as, an anti-CD56 or NKG2D antibody or aptamer).

The method can further comprise contacting the coated EVs/PNPs, with or without a targeting agent, with target cells. In one embodiment, the target cells are T cells. The coated EVs/PNPs and cells can be contacted in vitro, ex vivo, or in vivo.

Retroviral Gag proteins can be expressed in the producer cells to form a capsid that is released from the cell in an EV. In some embodiments, the capsids contain retroviral Gag and Pol proteins. Preferably, the capsids contain capsid, matrix, nucleocapsid, protease, reverse transcriptase, and integrase proteins.

In some embodiments, the retrovirus is an avian leukosis virus, Rous sarcoma virus, mouse mammary tumor virus, murine leukemia virus, feline leukemia virus, bovine leukemia virus, or Human T-lymphotropic virus. In some embodiments, the retrovirus is a lentivirus, such as human immunodeficiency virus type 1 (HIV-1), human immunodeficiency virus type 2 (HIV-2), simian immunodeficiency virus, or feline immunodeficiency virus.

Preferably, the capsids contain retroviral Gag and Pol proteins. Most preferably, the capsids contain capsid, matrix, nucleocapsid, protease, reverse transcriptase, and integrase proteins.

In some embodiments, the capsid contains a pair of RNAs. The RNAs preferably contain retroviral packaging signals. RNAs also preferably contain retroviral signals for reverse transcription.

In some embodiments, the RNAs encode proteins, preferably non-retroviral proteins. In some embodiments, the proteins are markers proteins, such as β-galactosidase, chloramphenicol acetyltransferase, green fluorescent protein, red fluorescent protein, and luciferase enzyme.

In some embodiments, the proteins are cytokines or growth factors, such as interferons, interleukins, lymphokines, and tumor necrosis factors, for example, IL-1, IL-2, IL-4, IL-6, IL-10, IL-11, IL-13, IL-17, IL-35, TNFa, IFN-α, IFN-β, IFN-γ, TGF-β, GM-CSF, M-CSF, G-CSF, EPO, BMP, EGF, FGF, and VEGF.

Such therapeutic proteins include chimeric antigen receptors and chimeric costimulatory receptors (CCRs). CCR are chimeric receptors that comprise an antigen-binding extracellular domain, a transmembrane domain and an intracellular signaling domain (Sadelain et al., Cancer Discov. 2013, 3 (4): 388-398) but that do not have a T cell activation domain, but do have a co-stimulatory domain, such as for example, CD28, 4-1BB, OX40, ICOS, DAPIO, 2B4, CD70, or the like. CCRs can be used in conjunction with a T cell receptor or a CAR to enhance T cell reactivity against dual-antigen expressing T cells. CCRs can also be used to enhance selective tumor targeting (Kloss et al., Nat Biotechnol, 2013, 31 (1): 71-5). A CCR is an antigen-specific co-stimulatory receptor, which mimics the affects 4-1BB, OX40, ICOS or CD70 (depending on the co-stimulatory domain of the CCR) upon binding to its binding partner, i.e., a target antigen.

Chimeric antigen receptors (CARs) include those directed against carbonic anhydrase IX, (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD117, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (ACHR), folate receptor-a, FLT3, Ganglioside G2 (GD2), Ganglioside G3 (GD3), human Epidermal Growth Factor Receptor 2 (HER-2), human telomerase reverse transcriptase (HTERT), Interleukin-13 receptor subunit alpha-2 (IL-13Ra2), K-light chain, kinase insert domain receptor (KDR), Lewis Y (LeY), L1 cell adhesion molecule (LICAM), melanoma antigen family A1 (MAGE-A1), Mucin 16 (MUC16), Mucin 1 (MUC1), Mesothelin (MSLN), ERBB2, MAGE-A3, MAGE-A4, p53, MART1, GP100, Proteinase3 (PR1), Tyrosinase, Survivin, hTERT, EphA2, NKG2D ligands, cancer testis antigen NY-ESO-1, oncofetal antigen (h5T4), prostate stem cell antigen (PSCA), prostate-specific membrane antigen (PSMA), ROR1, tumor associated glycoprotein 72 (TAG-72), vascular endothelial growth factor R2 (VEGF-R2), and Wilms tumor protein (WT-1), BCMA, NKCS1, EGFIR, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, B-cell activating factor receptor (BAFF-R), Thymic stromal lymphopoietin (TSLPR), SLAM Family Member 7 (SLAMF7) and G-protein coupled receptor family C group 5 member D (GPRC5D).

Preferably, the chimeric antigen receptor (CAR) is one directed against CD19, CD20, CD22, CD30, CD33, CD38, CD123, CD133, CD147, CD317, FLT3, BCMA, DR4, CS-1, CLL-1, ADGRE2, HER2, HSP70, VEGF, GD2, GD3, MUC1, MUC16, NKR2, NKG2D ligands, CYP1B1, SP17, PRAME, and WT1. Chimeric antigen receptors (CARs) are preferably those directed against CD19, present in B-cell-derived cancers such as acute lymphoblastic leukemia (ALL) and diffuse large B-cell lymphoma (DLBCL); CD30, present in refractory Hodgkin's lymphoma; CD33, CD123, ADGRE2 and FLT3, present in acute myeloid leukemia (AML); and BCMA, present in multiple myeloma.

### Polymers

The invention encompasses EVs, preferably shaved EVs, coated with polymers. The EVs can be coated with at least one polymer to form a polymeric nanoparticle.

The invention encompasses a method comprising a) providing EVs; b) treating the EVs with a protease to remove proteins on the outer surface of the EVs; c) purifying the treated (shaved) EVs from the protease; d) coating the treated (shaved) EVs with at least one polymer to form a polymeric nanoparticle and e) purifying the coated and treated (shaved) EVs from uncoated polymers.

The invention encompasses a method comprising a) providing EVs containing a capsid; b) treating the EVs with a protease to remove proteins on the outer surface of the EVs; c) purifying the treated (shaved) EVs from the protease; d) coating the treated (shaved) EVs with at least one polymer to form a polymeric nanoparticle and e) purifying the coated and treated (shaved) EVs from uncoated polymers.

Preferably, the polymeric nanoparticle comprises a targeting moiety, most preferably for T cells or NK cells (e.g., an CD3 or NKG2D targeting agent, such as, an anti-CD3 or an anti-NKG2D antibody or aptamer). The method can further comprise contacting the coated EVs/PNPs, with or without a targeting agent, with target cells. In one embodiment, the target cells are T cells or NK cells. The coated EV/PNPs s and cells can be contacted in vitro, ex vivo, or in vivo.

In various embodiments, the number of polymer molecules per vector particle is at least about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰ or in a range between any of these amounts (e.g., from about 10⁸ to 10¹⁰).

Polymers can be added to the EVs by mixing them together and allowing them to combine. Mixing can be facilitated using a vortex. In some embodiments, the mixture is allowed to combine for at least 1, 2, 5, 10, 20, 30, 45, or 60 minutes. In some embodiments, the mixture is allowed to combine for at least 1, 2, 3, 6, 12, 18, or 24 hours.

In some embodiments, the mixture is allowed to combine at about 1° C., 2° C., 4° C., 10° C., 15° C., 20° C., 25° C., 30° C., 35° C., 37° C., 40° C., 45° C., or 50° C., or in a range between any of these temperatures (e.g. from about 4° C. to 20° C.).

In some embodiments, the mixture is allowed to combine in pharmacopeia-approved buffer solutions compatible with systemic injections, such as Histidine (20 mM), Citrate Sodium Phosphate (10 mM), Succinate Sodium Phosphate (10 mM), Sodium Phosphate (10 mM), Citrate-Tris (10 mM), Acetate-Sodium Phosphate (10 mM), Acetate-Tris (10 mM) and Succinate-Tris (10 mM) all in a pH range from 5 to 7.4 and formulated with or without Sucrose (50 mM) and/or sodium chloride (175 mM).

### Coating of Shaved EV

### One Layer Coating

Polymer coating of shaved EVs can be performed as follows. The polymer is diluted in the same pharmacopeia-compatible formulation buffer used for the purified shaved EVs and briefly vortexed for homogenization. The polymer solution is added on the shaved vectors at an appropriate mass ratio to change the global charge of the particles and homogenization is performed by liquid phase mixing (e.g., vortexing or microfluidics). The complex is incubated at room temperature for 30 minutes before being used for analysis or transduction.

Polymer coating can be comprised of a mixture of free-polymer and polymer covalently-coupled to the targeting moiety.

### Layer-by-Layer Coating

Layer-by-layer method adds a shielding polymer to coated shaved EVs prepared with cationic polymers and by following the “one layer coating” method. To do so, excess polymer is removed from the formed polymeric nanoparticles (PNPs) using a hollow fiber tangential flow filtration step. This step allows the reformulation of coated nanoparticles in the pharmacopeia-compatible buffer that is used to dilute the shielding polymer. Shielding polymer solution is comprised of a mixture of free polymer and polymer covalently coupled to targeting moiety. After brief mixing (e.g., vortexing) for homogenization, the shielding polymer solution is added on the coated nanoparticle at an appropriate mass ratio to change the global charge of the particles and homogenization is performed by liquid phase mixing (e.g., vortexing or microfluidics). The complex is incubated at room temperature for 30 minutes before being used for analysis or transduction. Again, excess polymer is removed by hollow fiber tangential flow filtration step.

### Characterization Methods

Multi-Angle Dynamic Light Scattering (MADLS) can be used to compare global charge (zeta potential), size (average hydrodynamic diameter) and size distribution (Polydispersity index) of the shaved EVs before and after addition of polymers and thereby confirm coating efficiency. Freshly prepared coated EVs/PNPs can be diluted 16-(zeta potential and polydispersity index) or 20-fold (average hydrodynamic diameter) in final formulation buffer and homogenized by gentle vortexing before analysis on a ZETASIZER ULTRA instrument (Malvern Instruments).

### Transduction Efficiency by Flow Cytometry

Titration of PNPs can be performed as follows to confirm the restoration of infectivity after one layer or layer-by-layer coating methods on two different cell lines: HEK293T and Jurkat cells.

Four hours before transduction, HEK293T cells are seeded in 24-well plates at 8×10⁵ cells/well in DMEM containing 10% FBS, 1% Penicillin/Streptomycin (PS) and incubated at 37° C. and 5% CO₂. Just before transduction, Jurkat cells are seeded at 8×10⁵ cells/well in RPMI containing 10% FBS, 1% PS. Serial dilutions of shaved retroviral particles and PNPs (at a same physical titer) are incubated on cells at 37° C., 5% CO₂. After 2 hours, fresh culture medium is added into each well and incubation is extended to 3 days. At day 3, cells are collected (with trypsinization step for HEK293T), washed in DPBS 1X and directly fixed with fixation buffer (Biolegend, 420801) for GFP encoding vectors (SEQ ID NO: 3) and Zombie NIR for live/dead discrimination. For CD19-CAR encoding vector (SEQ ID NO 4), additional staining steps before fixation are performed by incubating first the CD19 CAR detection reagent (Miltenyi Biotec, 130-129-550) for 10 min, washed and the incubated with biotin antibody-VB515 (Miltenyi Biotec, 130-110-957).

The number of viable fluorescent-positive cells after transduction with shaved retroviral particles or PNPs is determined using a flow cytometer (ATTUNE NXT, Thermofisher).

qPCR Reactions for Infectious Titer Determination

Four hours before transduction, HEK293T cells are seeded in 24-well plates (Corning, 3513) at 8×10⁴ cells/well in DMEM (Gibco, 61965026) containing 10% FBS (Gibco, 10091148), 1% Penicillin/Streptomycin (PS) (Gibco, 15140122) and incubated at 37° C. and 5% CO₂. Just before transduction, Jurkat cells are seeded at 8×10⁴ cells/well in RPMI (Gibco, A1049101) containing 10% FBS, 1% PS. Cells are transduced with a minimum of 3 successive dilutions of shaved retroviral particles or PNPs and cultured for 72 h at 37° C. and 5% CO₂. After 72 hours, cells are harvested and pellets collected. Total genomic DNA from transduced cell-pellets is extracted using the Maxwell RSC Blood DNA kit (Promega, AS1400) according to manufacturer's instructions. Proviral DNA quantification is performed by qPCR using PERFECTA MultiPlex ToughMix (Quantabio, 95147-250). The amplification of integrated sequences is performed using the following LTR (long terminal repeat sequences) probe (5′-6FAM-AACCATTAGGAGTAGCACCCACCAAGG-BHQ1-3′ (SEQ ID NO: 5)) and primers (Forward: 5′-TGGAGGAGGAGATATGAGGG-3′ (SEQ ID NO: 6) and Reverse 5′ CTGCTGCACTATACCAGACA-3′ (SEQ ID NO: 7)). The analysis is performed using the QUANTSTUDIO 7 Pro (Applied Biosystems) to quantify the number of proviral copies per cells.

Preferably, the EVs are coated with one or two layers of polymers. In some embodiments, the polymer is a cationic, neutral or zwitterionic polymer, with or without a targeting agent. In some embodiments, the first layer of polymer is a cationic polymer and the second layer of polymer is anionic, neutral or zwitterionic polymer, with or without a targeting agent.

Polymers include polyarginine, polylysine, PEI, chitosan, PBAE, PEI-co-polyhistidine, polylysine dendigraft/dendrimer/branched polymer, star shaped polyaminoacid based polymer, chondroitin sulfate, heparosan, polyglutamic acid, hyaluronic acid, or dextran sulfate, polysarcosine, PEG, polycarboxybetaine, polysulfobetaine or polyphosphorylcholine.

The invention encompasses EVs comprising cationic polymers. Cationic polymers include Poly amino Acids, Polyarginine, Polylysine, Polyether Imine (PEI), Chitosan, Poly(β-amino ester) (PBAE), Polyethylene Imine/polyTetra Hydo Furan (PEI/THF), OM-PBAE, and Dendrigrafts of Lysine (DGL), and modifications thereof.

Anionic polymers include Chondroitin sulfate, Sodium Hyaluronate, Heparosan, Poly Glutamic Acid (PGA), Alginate, Dextran sulfate. Cationic and anionic polymers can be used with shielding agents, which include PolySarcosine, Polyzwitterion and Polyethyleneglycol (PEG), polycarboxybetaine, polysulfobetaine or polyphosphorylcholine.

Zwitterion polymers comprise a positively charged nitrogen atom and a negatively charged oxygen atom, separated each other by at least one carbon atom, preferably from two to three carbon atoms. Zwitterion polymers include polycarboxybetaine, polysulfobetaine and polyphosphorylcholine.

In some embodiments, the zwitterion is a fusion of an anionic or cationic with a zwitterion.

### Targeting Agents

The invention encompasses EVs comprising targeting agents. The targeting agents can direct the EVs to specific cellular targets.

In various embodiments, targeting agents comprise antibodies, including human, mouse, camelid and shark antibodies, single chain antibodies, nanobodies, diabodies, and antigen-binding fragments of antibodies, peptides, nucleic acids such as aptamers or oligonucleotides, and ligands that bind to cell-surface receptors or components of extracellular matrix.

In one embodiment, the targeting agent is a CD3 targeting agent, for example, an anti-CD3 antibody or aptamer. In various embodiments, the targeting agent is an anti-NKG2D antibody or aptamer.

The aptamer can be any of the aptamers disclosed in Riccardi et al., Molecules 2020, 25, 5227, 1-34, which is hereby incorporated by reference.

The invention encompasses methods of adding targeting agents to EVs. In various embodiment, the targeting agent (e.g., aptamer) is a charged molecule (e.g., negatively charged) that can interact with and be incorporated into the polymeric nanoparticle.

The targeting agent can be covalently grafted on the polymer (the second layer or the one that will be in contact with the extracellular medium). A mix of targeted and non-targeted polymers can be used to coat the EV.

### Method of Delivering Payloads

The EVs of the invention can be used as delivery vehicles for payloads, such as therapeutic proteins and nucleic acids encoding them.

In various embodiments, the invention comprises a method comprising contacting EVs in vitro, ex vivo, or in vivo with eukaryotic cells.

The invention encompasses a method comprising a) providing polymeric nanoparticles comprising shaved EVs containing a payload; and b) contacting the polymeric nanoparticles (PNPs) with eukaryotic cells.

The invention encompasses a method comprising a) providing shaved EVs containing a payload; b) coating the treated EVs with at least one polymer to form a polymeric nanoparticle; and c) contacting the polymeric nanoparticles (PNPs) with eukaryotic cells.

The invention encompasses a method comprising a) providing EVs containing a payload; b) treating the EVs with a protease to remove proteins on the outer surface of the EVs; c) purifying the treated EVs from the protease; d) coating the treated EVs with at least one polymer to form a polymeric nanoparticle (PNP); and e) contacting the PNPs with eukaryotic cells. In some embodiments, the PNPs are purified prior to contacting them with eukaryotic cells.

Preferably, the EVs comprise a retroviral capsid. The retroviral capsid can contain the retroviral proteins necessary for reverse transcription, with or without the ability for integration. More preferably, the capsid comprises RNA molecules encoding a protein, preferably a therapeutic protein (e.g. a CAR).

Preferably, the polymeric nanoparticle comprises a targeting moiety, most preferably for T cells or NK cells (e.g., an CD3 or NKG2D targeting agent, such as, an anti-CD3, an anti-NKG2D antibody or aptamer). The method can further comprise contacting the coated EVs/PNPs, with or without a targeting agent, with target cells. In one embodiment, the target cells are T cells or NK cells. The coated EVs/PNPs and cells can be contacted in vitro, ex vivo, or in vivo.

In one embodiment, the cells are T cells or NK cells and the payload is a pair of RNAs encoding a CAR.

In various embodiments, the ratio of EVs (or PNPs) to eukaryotic cells can be about 1:100, 1:10, 1:1, 10:1, 100:1, 103:1, 104:1, 105:1, or 106:1, or in a range between any of these ratios (e.g., from about 100:1 to 104:1).

In various embodiments, 10⁴, 10⁵, 10⁶, 10⁷, 10⁸, 10⁹, 10¹⁰, 10¹¹, 10¹², 10¹³, 10¹⁴, 10¹⁵, etc. EVs (or PNPs), preferably shaved EVs, can be contacted with 10⁴, 10⁵, 10⁶, 10⁷, 10⁸, 10⁹, 10¹⁰, 10¹¹, 10¹², 10¹³, 10¹⁴, 10¹⁵, etc. cells, preferably T cells, or any ranges between any of these amounts (e.g., from 10⁷ to 10¹⁰ EVs with from 10⁵ to 10⁸ cells).

In one embodiment, the method comprises contacting 10⁴, 10⁵, 10⁶, 10⁷, 10⁸, 10⁹, 10¹⁰, 10¹¹, 10¹², 10¹³, 10¹⁴, 10¹⁵, etc. EVs (or PNPs) ex vivo with 10⁴, 10⁵, 10⁶, 10⁷, 10⁸, 10⁹, 10¹⁰, 10¹¹, 10¹², 10¹³, 10¹⁴, 10¹⁵, etc. T cells or NK cells from a patient and reintroducing the T cells or NK cells into the patient.

In some embodiments, the eukaryotic cells are from an animal, such as avian or mammalian sources, such as chicken, mouse, primate, monkey, human, hamster, rat, etc.

Cells include immune cells and fibroblasts. In some embodiments, the cells are peripheral blood mononuclear cells, B cells, NK cells, T cells, macrophages, dendritic cells, stem cells, neurons, or iPSCs.

Isolated T cells and NK cells can be prepared using well known techniques, for example, using paramagnetic polystyrene beads coated with anti-CD3 and anti-CD28 monoclonal antibodies as described in Laport et al., Blood 2003, 102:2004-2013. Contacting cells with EVs containing capsids can be performed as described in Levine et al., PNAS 2006, 103:17372-17377.

The invention further encompasses cells comprising the EVs, capsids, or payloads of the invention.

### EXAMPLES

### Example 1—Production of EVs

### A. Without Capsids

Extracellular Vesicles were produced in HEK293 derived cells (Gibco Invitrogen) in suspension. HEK293 derived cells were seeded at 3.5×10⁵ cells/mL in glass bioreactors in 1000 mL of LV-MAX™ Production Medium (Gibco Invitrogen). After 5 days incubation at 37° C. with constant magnetic stirring under humidified 8% CO₂, bulk mixture was harvested by centrifugation at 2000 g for 15 min to remove cells and cell debris in the supernatant and was clarified by filtration through 0.45 μm and 0.22 μm PVDF membranes (STERICUP, Merk Millipore).

B. Containing capsids

EVs containing capsids were produced in HEK293 derived cells (Gibco Invitrogen) in suspension by using LV-MAX™ reagent-mediated transient transfection of 293F suspension cells with third generation Gag-Pol, Rev packaging and VSV-G envelope plasmids, and GFP-or CD19 CAR encoding transfer plasmid. See e.g., Perry et al., Viruses 2021, 13:268.

HEK293 derived cells were seeded at 3.5×10⁶ cells/mL in 8×1000 mL Erlenmeyer flasks (Corning) in 204 mL of LV-MAX™ Production Medium (Gibco Invitrogen). The Erlenmeyers were incubated at 37° C., 120 rpm under humidified 8% CO₂. The day after seeding, the transient transfection was performed at a cell density of 4×10⁶ cells/mL. LV-MAX™ Transfection Reagent (Gibco Invitrogen) was mixed with GFP or CD19 CAR transfer, Gag-Pol packaging, Rev packaging and VSV-G envelope plasmids. After 10 min incubation at room temperature, the transfection mix/plasmid complex was slowly added to the cells and incubated at 37° C., 120 rpm under humidified 8% CO₂. 6 hours post-transfection, the production of EVs containing capsids was stimulated by adding LV-MAX™ Enhancer (Gibco Invitrogen). The bulk mixture was incubated at 37° C., 120 rpm under humidified 8% CO₂ for 48 hours. One hour before clarification, 5 U/mL of DENARASE in 2 mM MgCl₂ (c-LEcta GmbH) were added and DNAse treatment was performed at 37° C., 120 rpm under humidified 8% CO₂. Bulk mixture was harvested by centrifugation at 1300 g for 15 min to remove cells and cell debris in the supernatant and was clarified by filtration through 0.45 μm PVDF membranes (STERICUP, Merk Millipore).

### Example 2—Purification of EVs

### A. Without Capsids

Purification of EVs was performed by multimodal chromatography on a CAPTO Core 700 membrane (Cytiva). Tangential flow filtration was performed on a 300 kDa MIDIKROS MPES membrane (Repligen), which allowed to reduce the volume and to formulate in pharmacopeia-compatible formulation. After sterile filtration at 0.22 μm (Millipore), final product was filled in 2 mL cryotubes with aliquots less than 1 mL, then labelled, frozen and stored at <−70° C.

TABLE 1
Characteristics of EV batches produced from HEK293
derived cells. Physical titers are measured
by NTA (Nanotracking analyzer) and MADLS.

| | | Physical Titer | Physical Titer |
|---|---|---|---|
| EV batch | Final Volume | NTA | MADLS |
| # | (mL) | (particle/mL) | (particle/mL) |
| 1 | 2 | 2.6 × 10¹¹ | NA |
| 2 | 0.1 | 2.6 × 10¹⁰ | 1.28 × 10¹⁰ |
| 3 | 0.1 | 2.55 × 10¹⁰ | 3.36 × 10¹⁰ |
| 4 | 0.8 | 3.4 × 10¹⁰ | 2.70 × 10¹⁰ |
| 5 | 14.5 | 1.4 × 10¹¹ | 1.50 × 10¹¹ |
| 6 | 30 | 8.25 × 10¹⁰ | 8.47 × 10¹⁰ |

### B. Containing Capsids

Purification of EVs containing capsids was performed by anion exchange chromatography on a Q mustang membrane (Pall Corporation) and stepwise NaCl elution. Tangential flow filtration was performed on a 500 kDa MIDIKROS MPES membrane (Repligen), which allowed to reduce the volume and to formulate in pharmacopeia-compatible formulation. Final product was filled in 2 mL cryotubes with aliquots less than 1 mL, then labelled, frozen and stored at <−70° C.

TABLE 2
Characteristics of LV batches produced from HEK293-derived cells.

| | | | | Infectious | | | | | |
|---|---|---|---|---|---|---|---|---|---|
| LV | | | Infectious | Titer | Physical | | | | |
| GFP | | Final | Titer | Flow | titer | | | Residual | |
| batch | | Volume | qPCR | cytometry | RT-qPCR | p24 | HCP | DNA | Endotoxin |
| # | GOI | (mL) | (TU/mL) | (TU/mL) | (copies/mL) | (particle/mL) | (ng/mL) | (ng/mL) | (EU/mL) |
| 1 | GFP | 29.0 | 5.48 × 10⁸ | 4.14 × 10⁸ | 1.34 × 10¹¹ | 2.22 × 10¹¹ | 12400 | 530 | 2.11 |
| 2 | GFP | 32.0 | 7.76 × 10⁸ | 6.58 × 10⁸ | 3.76 × 10¹¹ | 2.21 × 10¹¹ | 10550 | 570 | 1.3 |
| 3 | GFP | 28.5 | 1.17 × 10⁹ | 1.39 × 10⁹ | 3.80 × 10¹¹ | 6.95 × 10¹² | 18040 | 3350 | 17.6 |
| 4 | CD19 | 47 | 8.3 × 10⁷ | 1.19 × 10⁸ | 1.3 × 10¹¹ | 6.15 × 10¹² | 10750 | 920 | 11.9 |
| | CAR | | | | | | | | |

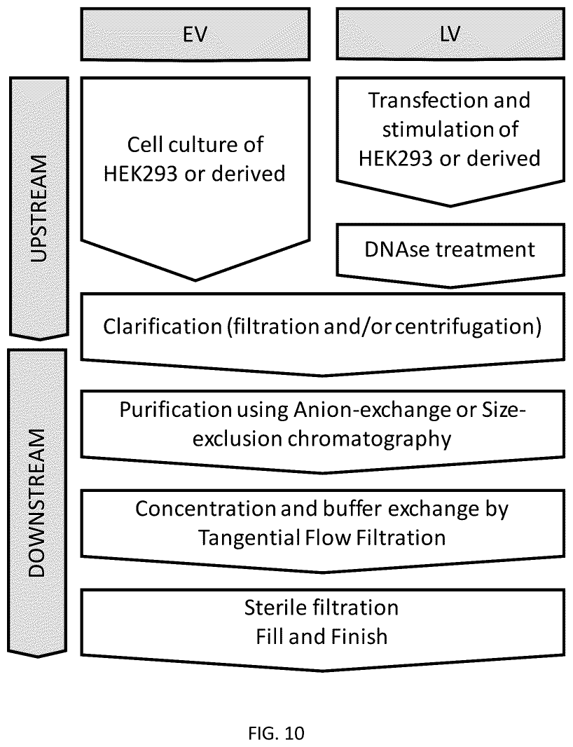

Similar bioproduction processes in HEK293-derived cells have been implemented to obtain EVs containing or not capsids with comparable biophysical and biochemical properties. These process steps that secure batch to batch consistency are summarized in FIG. 10.

### Example 3—Assay of Surface Proteins on EVs

### Multiplex Detection of Surface Proteins

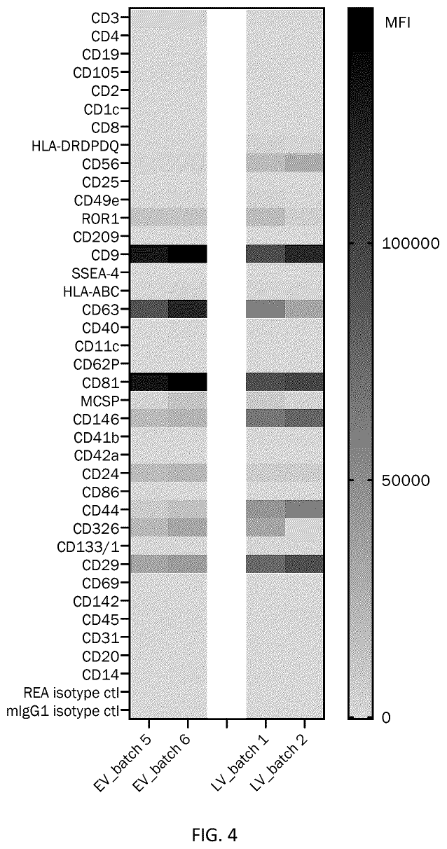

The MACSPLEX Exosome kit (Miltenyi Biotec) can be used to compare surface epitopes detected on vectors before and after shaving. The method allows the quantification of the expression of up to 37 human membrane epitopes using specific antibodies. The overnight protocol was followed according to manufacturer's instructions using 10⁷, 10⁸ or 10⁹ EV particles and detection with anti-CD9, or anti-CD63, or anti-CD81 or all three antibodies. As shown in FIG. 4, surface expression profiles of EVs without capsids (EVs) or EVs containing capsids (LVs) produced in HEK293T-derived cells are similar. Obtained results confirm that CD9, CD63 and CD81 are interesting markers present on both types of EVs.

### Direct and Sandwich ELISA

EVs produced in HEK293T or derived should express human Tetraspanins CD9, CD63 and CD81 at their surface and CD24, CD29, CD44, CD146 and CD346 membranes proteins. In addition, LVs can have a pseudotyping envelope, such as VSV-G proteins on their surface as well as transmembrane transgenes such as CAR and CCR.

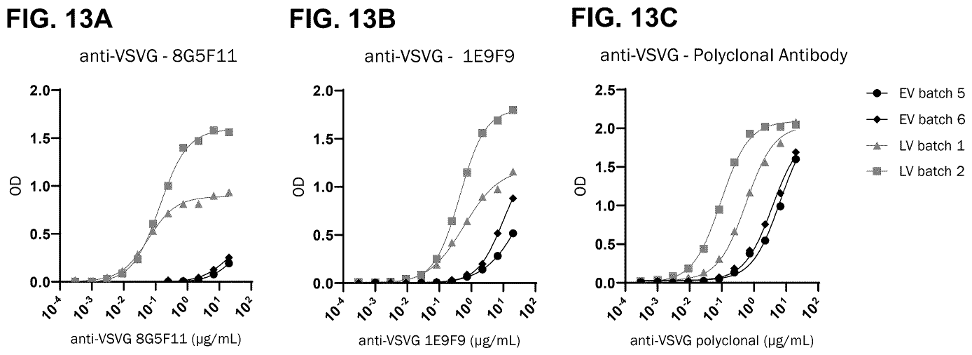

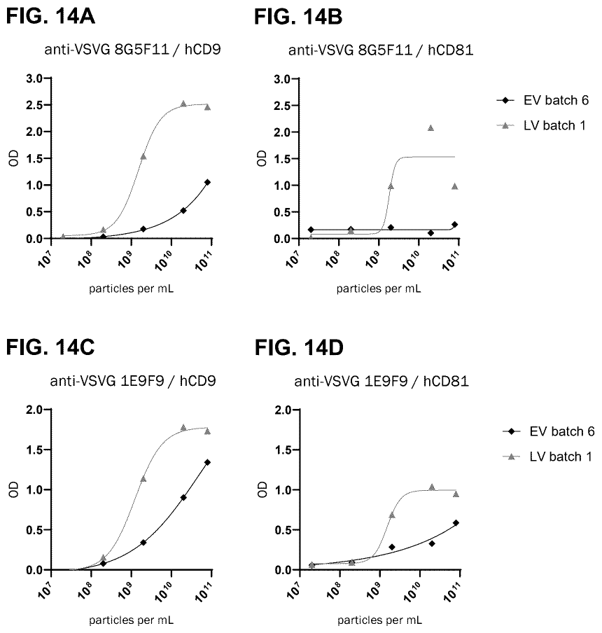

FIG. 13-14 show the results of an experiment in which unshaved EVs and LVs were analyzed by ELISA for the presence of VSVG. Titration was performed with anti-VSVG neutralizing monoclonal antibody 8G5F11, anti-VSVG monoclonal antibody 1E9F9 and anti-VSVG polyclonal antibodies and showed similar results.

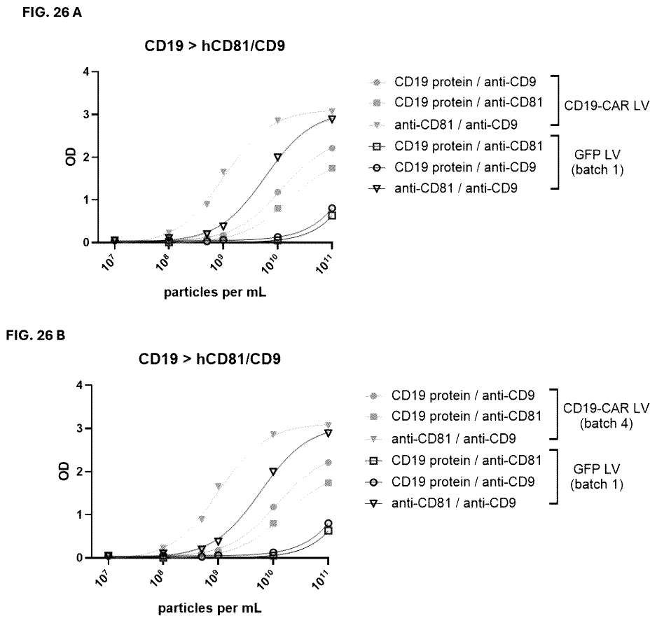

In addition, FIG. 26 shows the results of an experiment in which unshaved LV were analyzed by sandwich ELISA for the presence of CD19-CAR with CD19 recombinant proteins or anti-FMC63 idiotype antibody. CD19-CAR is detected on CAR-expressing LV but not GFP-expressing LV as expected.

Thus, shaving efficiency can be measured by the lack or decrease of binding of anti-CD9 (Biolegend, 312102, clone HI9a; Invitrogen, MA5-33125, clone 3A2), CD24 (Biolegend, 311102, clone ML5), CD29/Integrin beta 1 (Invitrogen, MA5-17103, clone 3B6), CD44 (Invitrogen, MA5-15462, clone 8E2F3), CD63 (Invitrogen, MA5-30187, clone 142; Biolegend, 353039, clone H5C6, CD81 (Biolegend, 349502, clone 5A6; Invitrogen, MA5-33123, clone 9F7), CD146 (Invitrogen, MA5-29414, clone 12), CD326 (Invitrogen, 14-9326-82, clone 1B7), VSVG (Sigma-Aldrich, MABF2337-100UG, clone 8G5F11; Sigma-Aldrich, MABF2321-100UL, 1E9F9) CAR (for FMC63 scFv: Miltenyi Biotec, 130-127-983, clone REA1297; Miltenyi Biotec, 130-127-984, clone REA1298) or CCR antibodies to the surface of EVs, or antigen like CD19 (Miltenyi, 130-129-550. ACROBiosystem, CD9-H52H2) to CAR and CCR, either by direct ELISA or sandwich ELISA.

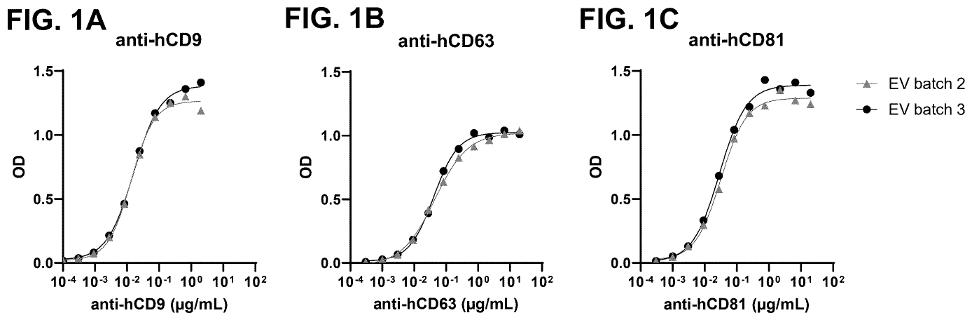

For direct ELISA, 108 EV per well were coated in a MAXISORP 96-well plate (Thermo Scientific, NUNC, 439454) overnight in DPBS 1X (Gibco, 14190094). After 2 hours saturation in 2% BSA (Thermo Scientific, 37525), a dilution range of primary antibody or antigen (listed above) was incubated for 1.5 hours. After washing with a solution of DPBS 1X and 0.05% Tween (Sigma, P9416), secondary antibodies or streptavidin/avidin coupled to HRP were incubated for 30 min. The HRP coupled reagents used were: goat anti-mouse IgG (H+L) HRP conjugate (Invitrogen, 31430) at 1/5 000, goat anti-mouse IgG (H+L) HRP conjugate (Invitrogen, 31460) at 1/10 000, Streptavidin-HRP (Thermo Scientific, 21130) at 1/10 000 or Avidin-HRP (Biolegend, 405103) at 1/5 000. After well washing, 1X TMB substrate solution (Invitrogen, 00-4201-56) was added for 10 min and stopped with a stop solution (Thermofisher, N600). Absorbance was measured using a GLOMAX Discover (Promega, GM3000) at 450 nm. FIG. 1A-C show titration curves obtained by direct ELISA with anti-CD9, anti-CD63 and CD81 antibodies on 2 independent batches of EVs not containing capsids.

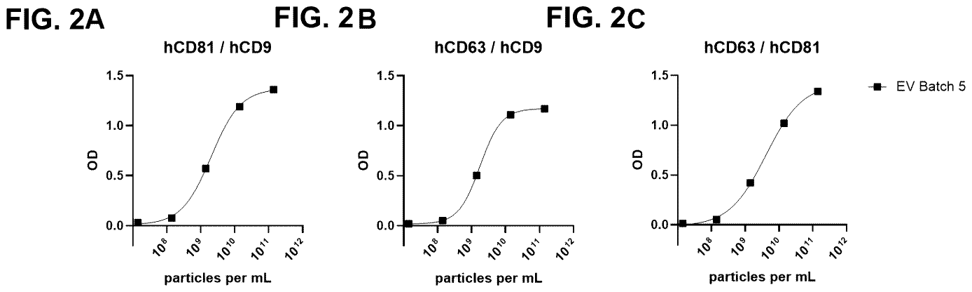

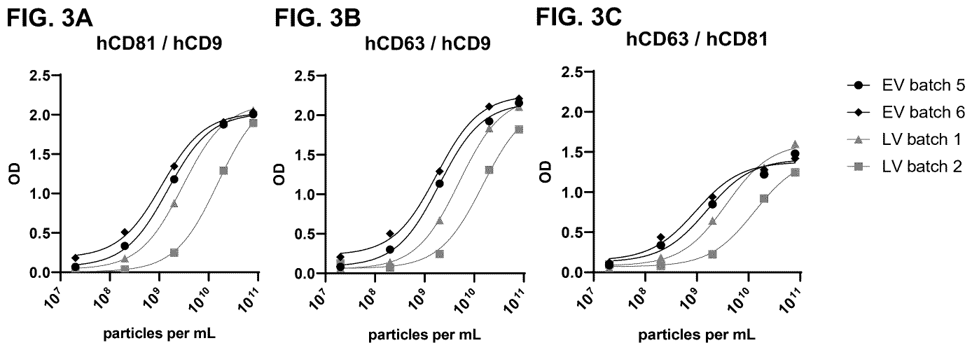

Antibody pairs have been selected for simultaneous detection of two different protein surface epitopes (tetraspanins, VSVG and/or CAR). Sandwich ELISA was performed with an overnight coating of a primary antibody, a saturation step and then an incubation of a range of EV for 2 hours at room temperature. A biotinylated antibody was incubated for 1.5 hours, followed by washing and incubation of streptavidin-HRP (Thermo Scientific, Pierce, 21130) at 1/10 000e dilution for 30 min. Absorbance was measured as described for direct ELISA. As shown in FIG. 2A-C anti-CD9, anti-CD63 and CD81 antibody pairs can be used in sandwich ELISA to evaluate the integrity of EVs not containing capsids. Titration profiles and sensitivity of the detection of sandwich ELISA are comparable when the methods are applied to EV containing or not capsids as shown in FIG. 3A-C.

### Example 4—Shaving of EVs

The extracellular parts of vectors surface proteins are digested by proteinase K or other broad spectrum proteases on purified or non-purified vectors.

The enzymatic treatment is performed by direct addition of recombinant proteinase K (Invitrogen, 25530015) into vectors samples at a final concentration of 0.01-100 μg/mL. The mixture is incubated at room temperature for up to 2 hours. Then, the proteolysis is stopped with PMSF (Thermo Scientific) or AEBSF (Thermo Scientific). The excess of PMSF or AEBSF ensures a complete inactivation of the enzyme before proceeding to purification and encapsulation.

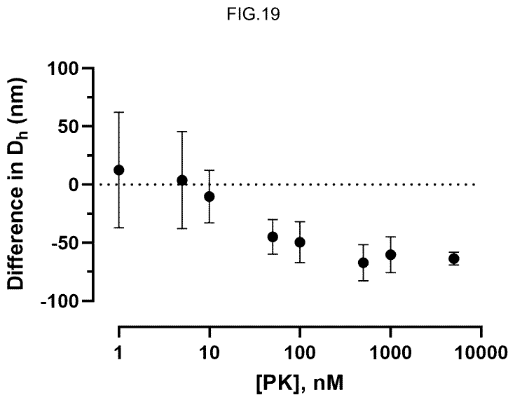

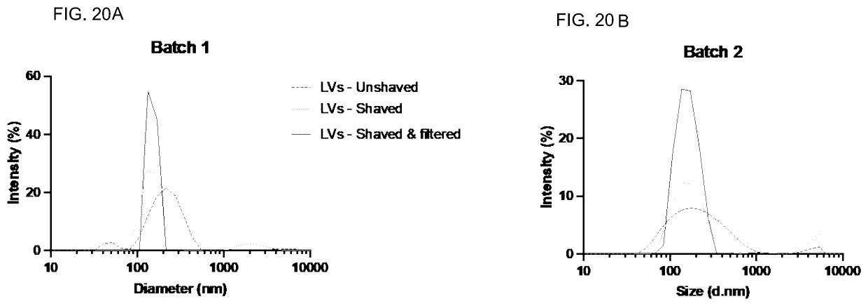

The size difference measured by Dynamic Light Scattering after Proteinase K (PK) treatment of 5.9×10⁹ LV particles produced in HEK293-derived cells with increasing concentrations of PK for 5 min at 25° C. and proteinase K inhibition with 20 mM AEBSF for 25 min was assessed. The results are shown in FIG. 19.

### Example 5—Purification of Shaved EVs

The removal of impurities after the proteolysis treatment is essential before polymer coating. The purification strategies used are based on the removal of impurities from the shaved EV using molecular weight/size-based methods.

For volumes of shaved EVs below 10 mL, the ultrafiltration method using PVDF or cellulose-centrifugal filter units (Millipore, AMICON ultra filter) of 100 to 500 kD molecular weight cut-off (MWCO) is used. The vector concentration step is followed by a diafiltration with at least 3 steps of 1/10e dilution for proper buffer exchange into the final polymer-coating buffer.

For larger volumes and increased purity, size exclusion chromatography (SEC), multimodal chromatography followed by a tangential flow filtration (TFF, hollow fibers or cassettes) or a TFF step alone is used to purify, concentrate and formulate in the specific polymer-coating buffer.

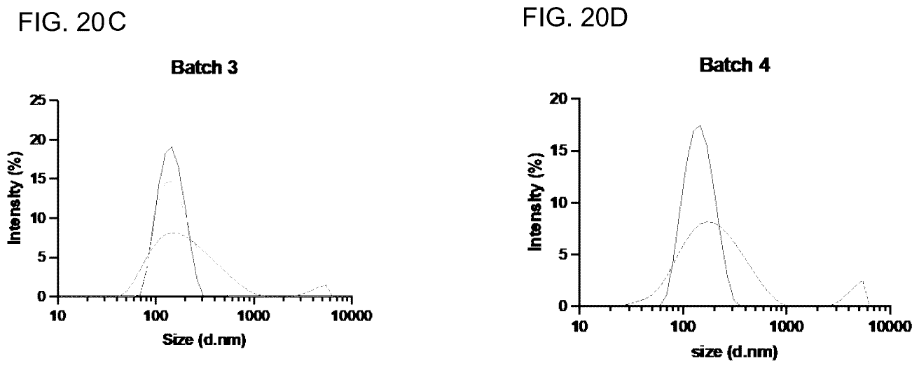

Four independent shaving reactions (Batch 1 panel A, batch 2 panel B, batch 3 panel C and batch 4 panel D) were performed on 1.43×10¹¹ LV particles with a 15 min incubation step with Proteinase K at 5 UM followed by 25 min inhibition with 20 mM AEBSF. Reaction products were filtered or not through a succession of 0.45 and 0.22 μm PVDF membranes. The size distribution of unshaved, shaved LV or shaved and filtered LVs produced in HEK293-derived cells measured by MADLS (Multiple Angle Dynamic Light Scattering). The results are shown in FIG. 20A-D.

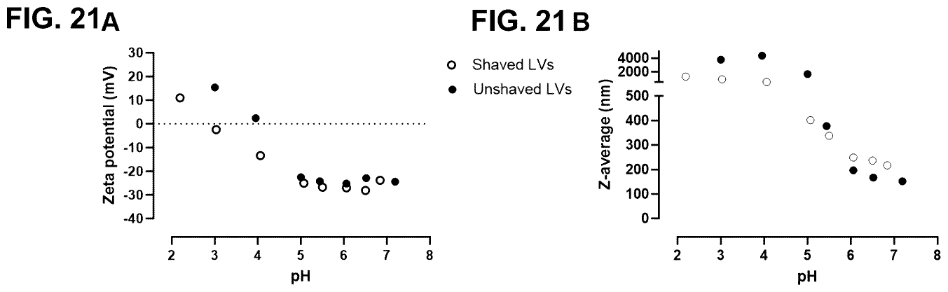

The variation of zeta potential and hydrodynamic diameter (z-average as a measure of the size as a function of the pH were determined for unshaved or shaved and filtered LVs particles by MADLS (Multiple Angle Dynamic Light Scattering). The results are shown in FIG. 21A-B.

### Example 6—Evaluation of the Shaving Process

### Multiplex Detection of Surface Proteins

The MACSPLEX Exosome kit (Miltenyi Biotec, 130-108-813) is used to compare surface epitopes detected on EVs before and after shaving. The method allows the quantification of the expression of up to 37 human membrane epitopes using specific antibodies. The overnight protocol is followed according to manufacturer's instructions.

### Direct and Sandwich ELISA

EVs produced in HEK293T or derived are expressing human tetraspanins CD9, CD63 and CD81 at their surface. In addition, lentiviral vector particles can harbor VSV-G and sometime CAR at their surface. Thus, shaving efficiency can be measured by the lack or decrease of binding of anti-CD9, CD63, CD81, VSVG and CAR antibodies and recombinant antigens to vectors, either by direct ELISA or sandwich ELISA.

For direct ELISA, 108 EVs per well are coated in a MAXISORP 96-well plate (Thermofisher, NUNC, 439454) overnight in DPBS 1X. After 2 hours saturation in 2% BSA, a dilution range of primary antibody is incubated for 1.5 hours followed by washing and incubation of secondary antibody coupled to HRP for 30 min. After well washing, 1X TMB substrate solution (eBiosciences, 00-4201-56) is added for 10 min and stopped with a stop solution (Thermofisher N600). Absorbance is measured using a GLOMAX DISCOVER (Promega, GM3000) at 450 nm.

Antibody pairs have been selected for simultaneous detection of two different protein surface epitopes (tetraspanins, VSVG and/or transgenes). Sandwich ELISA is performed with an overnight coating of a primary antibody, a saturation step and then an incubation of a range of vector for 2 hours at room temperature. A biotinylated antibody or antigen is incubated for 1.5 hours, followed by washing and incubation of streptavidin-HRP (Thermo Scientific, Pierce, 21130) at 1/10 000e dilution for 30 min. Absorbance is measured as described for direct ELISA.

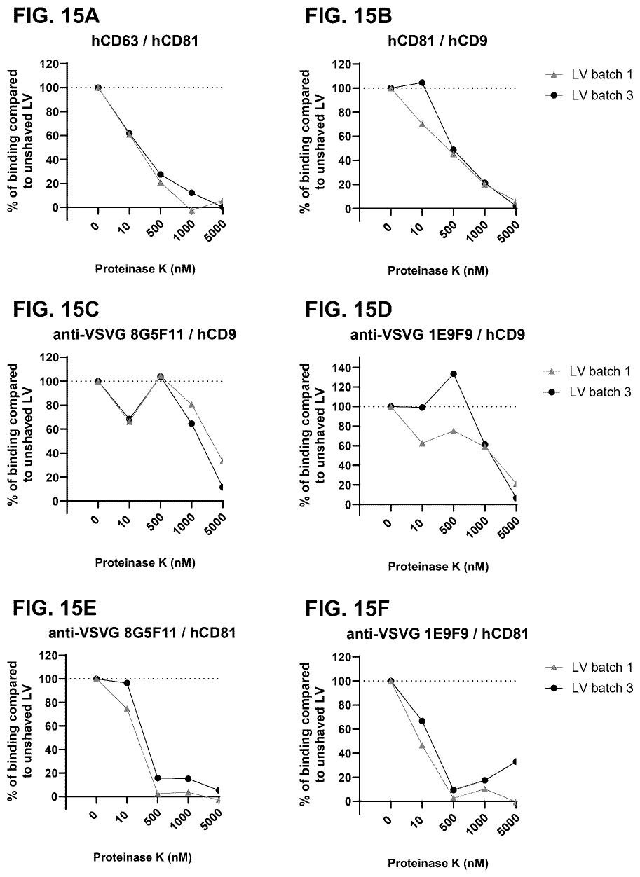

Shaving was performed on various LV batches for 15 min with 10, 500, 1000, and 5000 nM Proteinase K before 20 mM AEBSF treatment for 25 minutes. The resultant shaved EVs were then analyzed by being captured with anti-human CD63 and detected with anti-human CD81; captured with anti-human CD81 and detected with anti-human CD9; captured with anti-VSVG 8G5F11 and detected with anti-human CD9; D) captured with anti-VSVG 1E9F9 and detected with anti-human CD9; captured with anti-VSVG 8G5F11 and detected with anti-human CD81; or captured with anti-VSVG 1E9F11 and detection with anti-human CD81 and compared to unshaved LVs. The results are shown in FIG. 15. All of the markers used showed that shaving could remove nearly all of the tested surface markers. Shaving with 500 to 5000 nM Proteinase K was sufficient to remove nearly all of the surface proteins.

### Western Blot for Intra- and Extra-Cellular Domains Detection

Tetraspanins, VSVG, CAR and other proteins expressed at the surface of eVs are transmembrane proteins. Even if the surface domains are removed, their cytoplasmic domains are intact and not altered by the shaving procedure. Thus, EVs integrity after shaving can be assessed by western blot using specific antibodies targeting the cytoplasmic domains of the proteins CD9 (Invitrogen, MA5-33125, clone 3A2), CD63, CD81 (Abbomax, 630-910), CD326 (Abcam, ab233919, clone EGP40-1110), VSVG (Abcam, ab50549, clone P5D4), CD3 zeta (Sigma-Aldrich, MABF3032, clone 6B10.2) and CD28 (Invitrogen, MA5-36070, clone RM404; Invitrogen, PA5-116751). The EVs samples are first lysed in RIPA buffer (Thermo Scientific, 89900) or equivalent lysis buffer containing NP-40 or Triton to extract proteins from EV samples. Extracted and denatured EVs samples are loaded on NUPAGE 4-12% Bis-Tris gels (Thermofisher, NP0335BOX) and migration is performed in MES SDS running buffer (Thermofisher, NP0002) according to manufacturer's instructions. Then, the transfer is performed using the iBlot 2 dry blotting system (Thermofisher) on nitrate cellulose membrane (Invitrogen, IB23002). The blot is blocked in a solution of PBS 1X, 0.1% Tween 20 (PBST) and 3% BSA for 1 hour and washed in PBST three times. Then, primary antibodies are incubated in PBST with 0.2% BSA according to manufacturer's instructions, followed by three washes and incubation of HRP-conjugate reagent. Revelation is performed using the SUPERSIGNAL West Pico plus chemiluminescent substrate (Thermofisher, 34580) before acquisition on a IBRIGHT FL1500 Imaging system (Thermofisher, A44241).

### Transduction Efficiency

Titration of shaved vectors is performed as follows to confirm the loss of infectivity after shaving on two different cell lines: HEK293T and Jurkat cells.

Four hours before transduction, HEK293T cells are seeded in 24-well plates at 8×10⁵ cells/well in DMEM containing 10% FBS, 1% PS and incubated at 37° C. and 5% CO₂. Just before transduction, Jurkat cells are seeded at 8×10⁵ cells/well in RPMI containing 10% FBS, 1% PS. A serial dilution of unshaved and shaved vectors (at a same physical titer) is incubated on cells at 37° C., 5% CO₂. After 2 hours, fresh culture medium is added into each well and incubation is extended to 3 days. At day 3, cells are collected (with trypsinization step for HEK293T), washed in DPBS 1X and directly fixed with fixation buffer (Biolegend, 420801) for GFP encoding vectors. For CD19-CAR encoding vector, additional staining steps before fixation are performed by incubating first the CD19 CAR detection reagent (Miltenyi Biotec, 130-129-550) for 10 min, washed and the incubated with biotin antibody-VB515 (Miltenyi Biotec, 130-110-957).

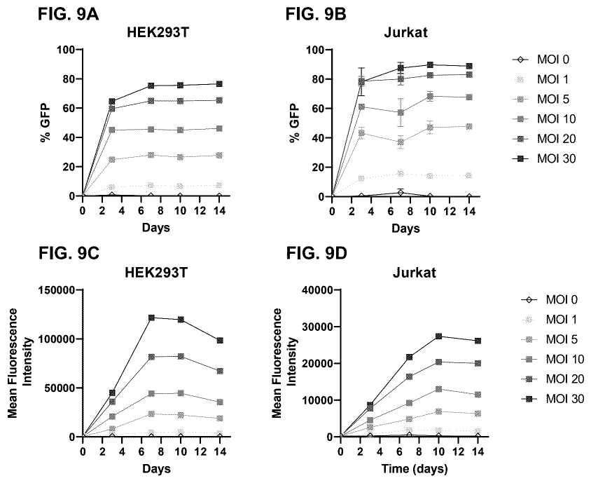

The number of fluorescent-positive cells after transduction with shaved or non-shaved vectors is determined using a flow cytometer (ATTUNE NXT, Thermofisher). As shown in FIG. 9, transduction efficiency of GFP encoding EVs containing capsids is dose-dependent and comparable in both cell models (irrespective of Mean Fluorescence Intensities or percentage of GFP cells readouts are considered). Maximum transgene expression is reached 3 days post-transduction and stays constant over 14 days, which confirms stable integration of the delivered transgene.

The genomic integration of the transgene delivered with shaved EVs, unshaved EVs, or PNPs is quantified by qPCR. HEK293T cells or Jurkat Cells are seeded in 24-well plates at 8×10⁴ cells/well. Cells are transduced with a minimum of 3 successive dilutions of shaved EVs, unshaved EVs, or PNPs and incubated for 72 h at 37° C. and 5% CO₂. After 72 hours, cells are harvested and pellets collected. Total genomic DNA from transduced cell-pellets is extracted using the MAXWELL RSC BLOOD DNA kit (Promega). Proviral DNA quantification is performed by qPCR using PERFECTA MULTIPLEX TOUGHMIX (Quantabio).

The amplification of integrated sequences is performed using probe and primers specific to the LTR (long terminal repeat sequences).

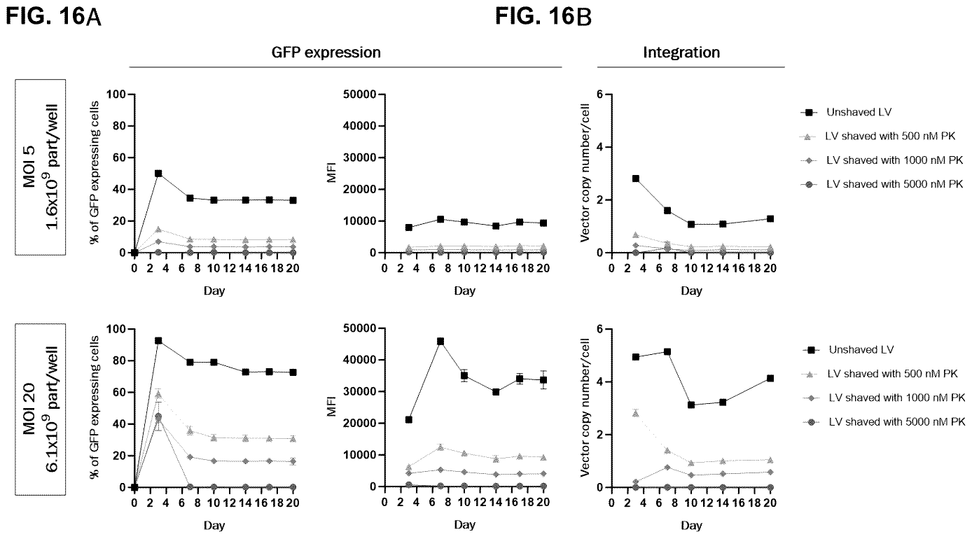

Shaved LV samples and unshaved LV were used to transduce Jurkat cells at various particles concentration: 1.6e9 particles equivalent to MOI 5 and 6.1e9 particles equivalent to MOI 20. GFP expression was monitored by flow cytometry from day 3 to day 20 post-transduction with the percentage of GFP expressing cells and the mean fluorescence intensity (MFI) of GFP. Vector copy number was quantified by qPCR on DNA extracted from transduced cells from day 3 to day 20. The results are shown in FIG. 16. Shaving with 500 to 5000 nM Proteinase K was sufficient to reduce both GFP expression and proviral DNA integration, with undetectable levels of expression and integration at 5000 nM Proteinase K.

For Proteinase K, 20 mg/mL=692 μM. Thus, 500 nM=14.45 μg/mL; 1000 nM=28.9 μg/mL; and 5000 nM=144.5 μg/mL.

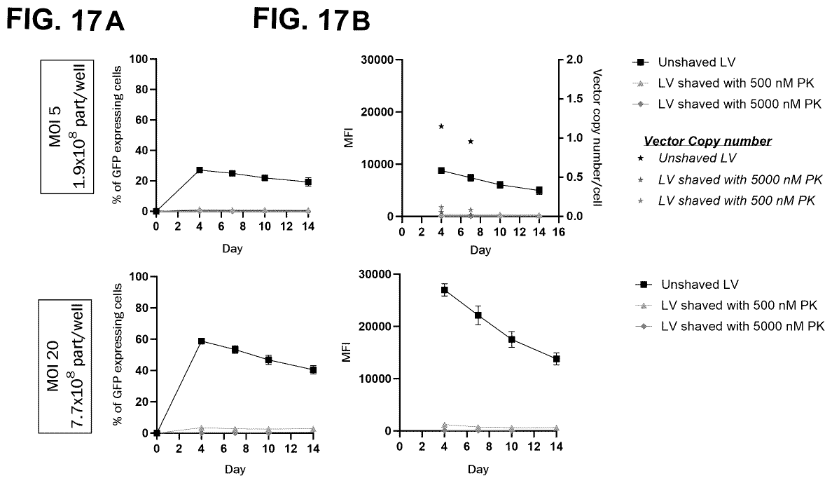

The transduction efficiency of shaved and unshaved LV in primary activated human T cells was similarly assessed. The results are shown in FIG. 17. Shaving with 500 to 5000 nM Proteinase K was sufficient to remove reduce both GFP expression and proviral DNA integration, with undetectable levels of expression and integration at 5000 nM Proteinase K.

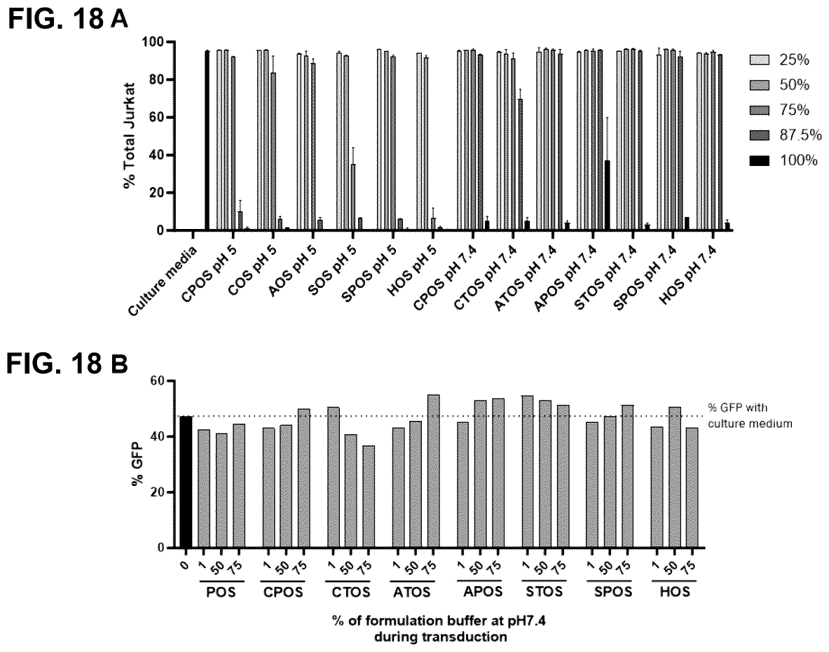

The impact of formulation buffer on Jurkat cells viability and LV transduction efficiency was assessed in different formulation mediums at pH5 or pH7.4. The results are shown in FIG. 18.

### Physical Titer

The physical titer quantification allows the titration of non-infectious shaved lentiviral vector. Three orthogonal methods are used based on the particles number (NTA), the quantification of capsid P24 protein or the quantification of viral RNA copy (RT-qPCR).

The particle concentration of shaved EVs can be determined by Nanoparticle Tracking analysis using a ZETAVIEW instrument (Particle Metrix). Before measurement, freshly prepared or frozen shaved EVs can be diluted 1000-fold in DPBS-50 mM Sucrose and homogenized by gentle vortexing.

The detection and quantification of p24 capsid vector protein is performed with the HIV-1 p24 antigen ELISA 2.0 kit (Zeptometrix, 0801002) according to manufacturer's instructions.

The quantification of proviral RNA copies is performed with the LENTI-X QRT-PCR titration kit (Takara, 631235) according to manufacturer's instructions after the isolation and purification of viral RNA with the MAXWELL RSC TNA Viral kit (Promega, AS1330).

### Example 7—Coating of EVs with Polymers

### One Layer Coating

Polymer coating of shaved vectors can be performed as follows. The polymer is diluted in the same pharmacopeia-compatible formulation buffer used for the purified shaved vectors and briefly vortexed for homogenization. The polymer solution is added on the shaved vectors at an appropriate mass ratio to change the global charge of the particles and homogenization is performed by liquid phase mixing (e.g., vortexing or microfluidics). The complex is incubated at room temperature for 30 minutes before being used for analysis or transduction.

Polymer coating can be comprised of a mixture of free-polymer and polymer covalently-coupled to the targeting moiety.

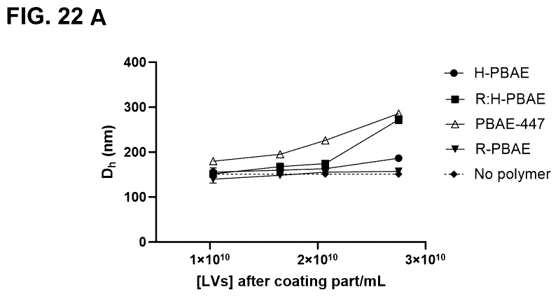

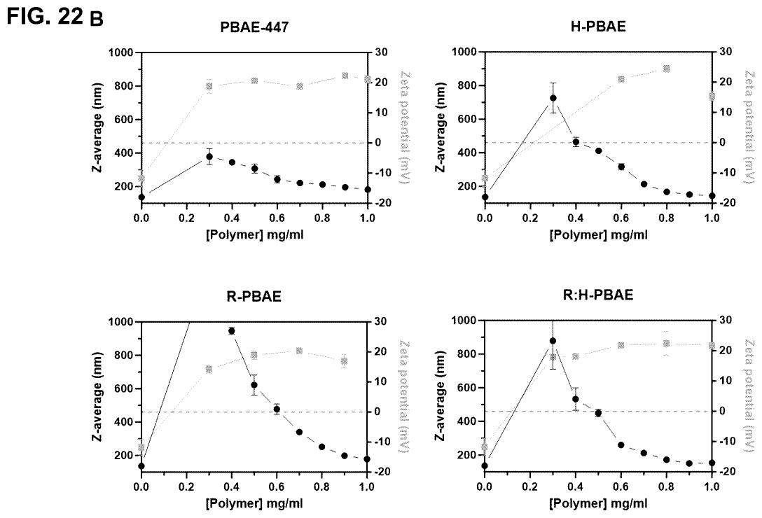

Viral particles and cationic polymers were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering). The variation of hydrodynamic diameter (z-average as a measure of the size) and zeta potential of polymeric nanoparticles was determined as a function of the viral particle concentration or cationic polymers concentration for shaved and filtered LVs particles produced in HEK293-derived cells. The results are shown in FIG. 22A-B.

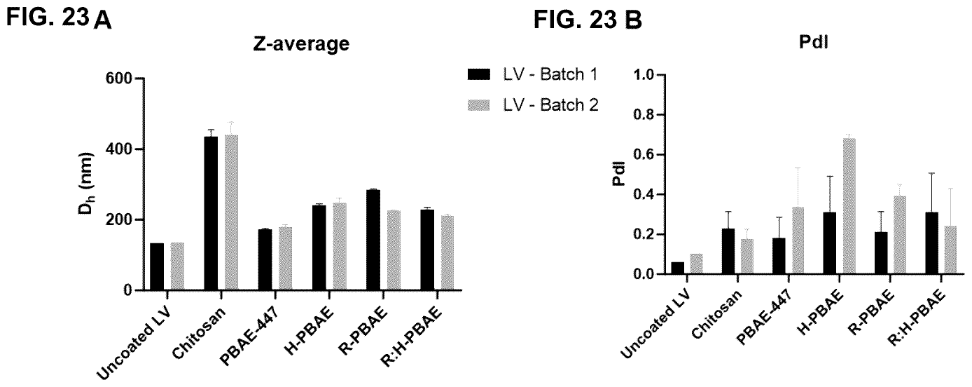

Viral particles and cationic polymers were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering). The variation of hydrodynamic diameter (z-average as a measure of the size) and size distribution (Pdl for Index of Polydispersity) of polymeric nanoparticles obtained from two different batches of shaved (with 5000 nM Proteinase K) and filtered LVs particles produced in HEK293-derived cells was determined. The results are shown in FIG. 23A-B.

### Layer-by-Layer Coating

Layer-by-layer method adds a shielding polymer to coated shaved vectors prepared with cationic polymers and by following the “one layer coating” method. To do so, excess polymer is removed from the formed polymeric nanoparticles using a hollow fiber tangential flow filtration step. This step allows the reformulation of coated nanoparticles in the pharmacopeia-compatible buffer that is used to dilute the shielding polymer. Shielding polymer solution is comprised of a mixture of free polymer and polymer covalently coupled to targeting moiety. After brief mixing (e.g., vortexing) for homogenization, the shielding polymer solution is added on the coated nanoparticle at an appropriate mass ratio to change the global charge of the particles and homogenization is performed by liquid phase mixing (e.g., vortexing or microfluidics). The complex is incubated at room temperature for 30 minutes before being used for analysis or transduction. Again, excess polymer is removed by hollow fiber tangential flow filtration step.

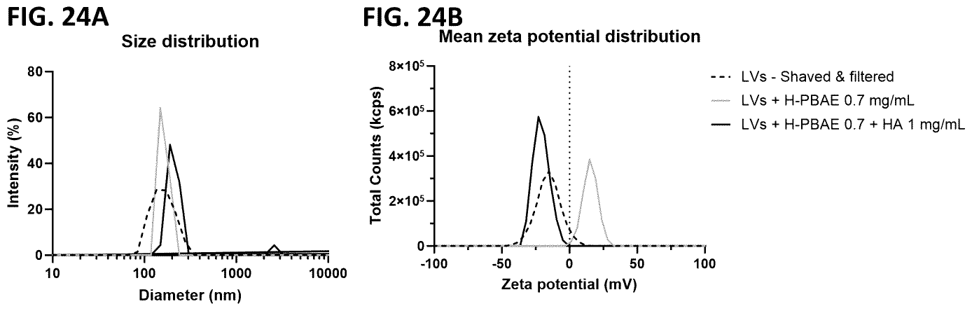

Viral particles, H-PBAE (cationic polymer) and HA (anionic polymer) polymers were diluted in 20 mM Histidine, 50 mM Sucrose, 127 mM NaCl PH 5 buffer. LVs were added to polymers and coating reaction was performed 30 min at 25° C. before measurement by MADLS (Multiple Angle Dynamic Light Scattering). Nanoparticles obtained after first coating step were added to anionic HA polymers and coating reaction was performed 30 min at 25° C. before measurement by MADLS. The results are shown in FIG. 24A-B.

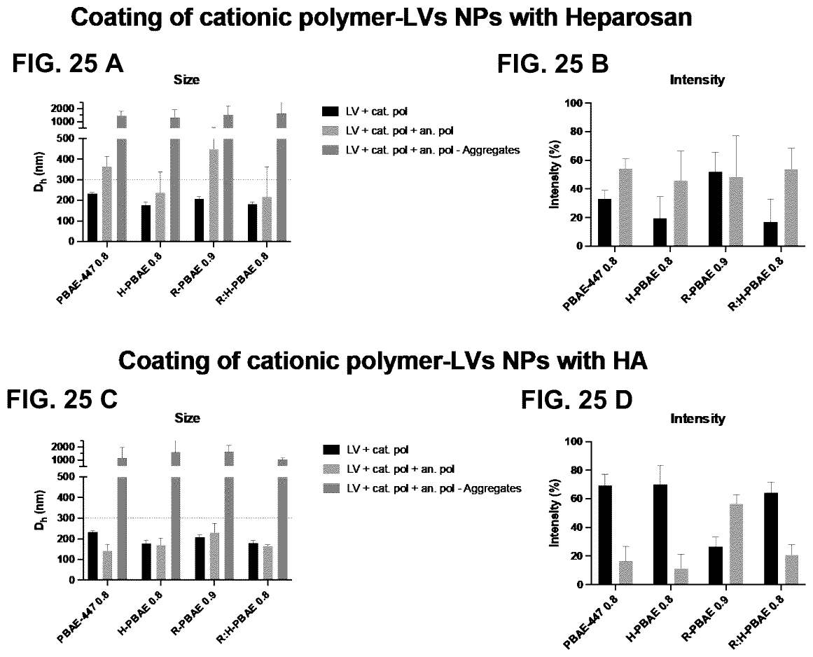

Viral particles, cationic (PBAE-447. H-PBAE and R-PBAE) and anionic polymers (Heparosan and HA) were diluted in 20 mM Histidine, 50 mM Sucrose, 127 mM NaCl pH 5 buffer. LVs were added to polymers and coating reaction was performed 30 min at 25° C. before measurement by MADLS (Multiple Angle Dynamic Light Scattering). Nanoparticles obtained after first coating step were added to Heparosan or HA anionic polymers and coating reaction was performed 30 min at 25° C. before measurement by MADLS. The results are shown in FIG. 25A-D.

### Characterization Methods

Multi-Angle Dynamic Light Scattering (MADLS) can be used to compare global charge (zeta potential), size (average hydrodynamic diameter) and size distribution (Polydispersity index) of the shaved EVs before and after addition of polymers and thereby confirm coating efficiency. Freshly prepared coated vectors can be diluted 16-(zeta potential and Polydispersity index) or 20-fold (average hydrodynamic diameter) in final formulation buffer and homogenized by gentle vortexing before analysis on a ZETASIZER ULTRA instrument (Malvern Instruments).

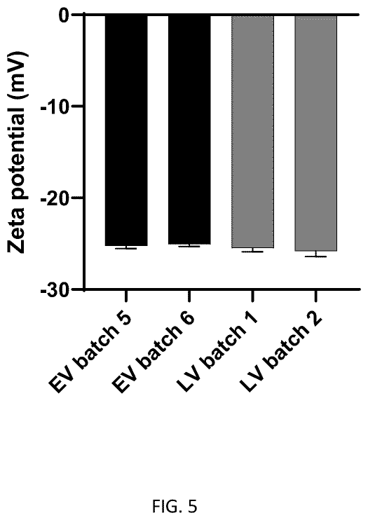

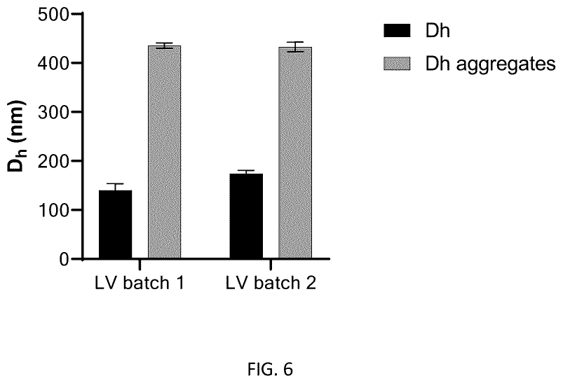

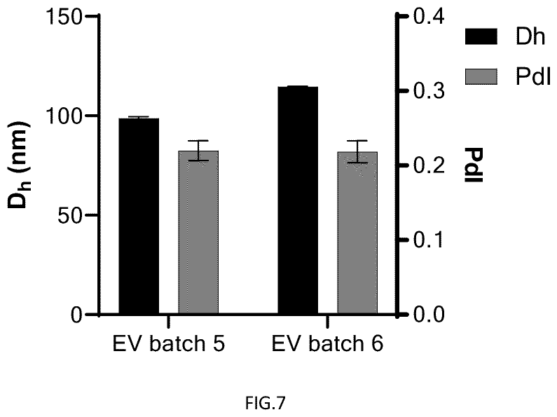

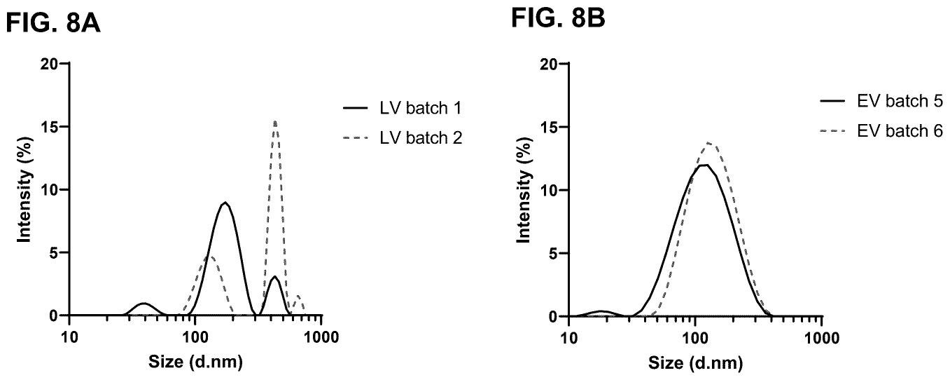

EVs containing or not capsids have similar global charges (cf FIG. 5), sizes (cf FIGS. 6 and 7) and size distributions (cf FIGS. 7 and 8). These biophysical properties are conserved batch after batch which is another proof of the robustness of the biomanufacturing processes that have been implemented with HEK293-derived cells.

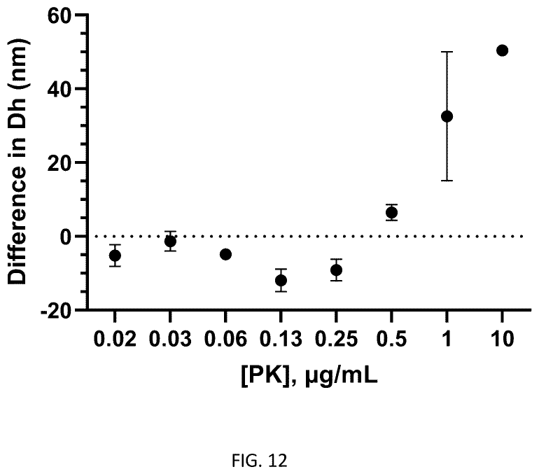

As shown in FIG. 12, Proteinase K treatment of EVs not containing capsids reduces the size of the vector by 10% due to the removal of extracellular parts of proteins expressed on the surface of EVs. Surprisingly, this modification only occurs in a limited range of protease concentration (0.03-0.3 μg/mL). Excess of protease induces the formation of EVs of higher sizes, most probably due to the formation of protein/particle aggregates.

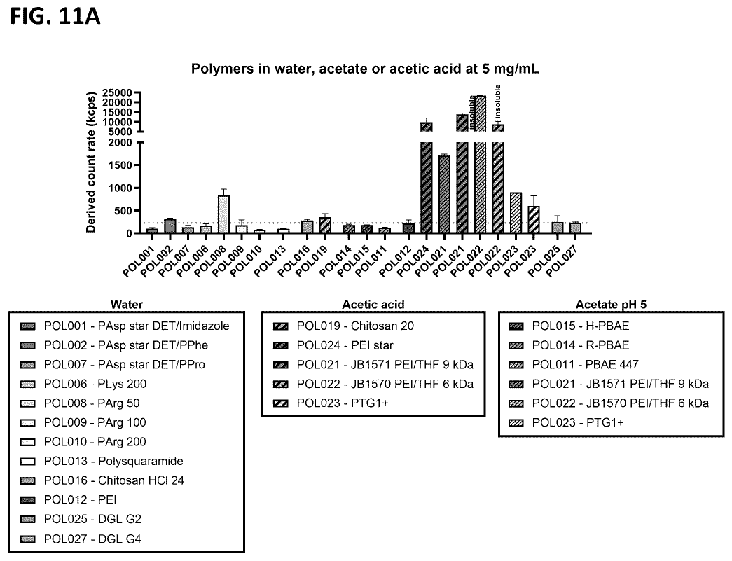

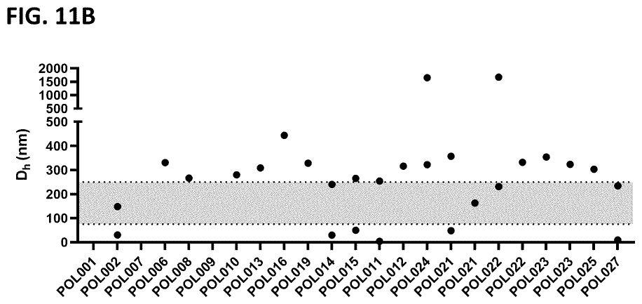

Because shaving process modifies biophysical properties of vectors, it is of primary importance to tightly control the coating reaction and the behavior of polymers in solution. As shown in FIG. 11, cationic polymers can form auto-assemblies when exposed to pharmacopeia-compatible formulation buffers. Therefore, coating conditions for shaved EVs must generate nanoparticles with size, size distribution and zeta potential easily distinguishable from polymers in excess that can then be easily removed from the formulated product.

### Transduction Efficiency

Titration of polymeric nanoparticles can be performed as follows to confirm the restoration of infectivity after one layer or layer-by-layer coating methods on two different cell lines: HEK293T and Jurkat cells.

Four hours before transduction, HEK293T cells are seeded in 24-well plates at 8×10⁵ cells/well in DMEM containing 10% FBS, 1% Penicillin/Streptomycin (PS) and incubated at 37° C. and 5% CO₂. Just before transduction, Jurkat cells are seeded at 8×10⁵ cells/well in RPMI containing 10% FBS, 1% PS. A serial dilution of shaved and shaved and coated vectors (at a same physical titer) is incubated on cells at 37° C., 5% CO₂. After 2 hours, fresh culture medium is added into each well and incubation is extended to 3 days. At day 3, cells are collected (with trypsinization step for HEK293T), washed in DPBS 1X and directly fixed with fixation buffer (Biolegend, 420801) for GFP encoding vectors and Zombie NIR for live/dead discrimination. For CD19-CAR encoding vector, additional staining steps before fixation are performed by incubating first the CD19 CAR detection reagent (Miltenyi Biotec, 130-129-550) for 10 min, washed and the incubated with biotin antibody-VB515 (Miltenyi Biotec, 130-110-957).

The number of viable fluorescent-positive cells after transduction with shaved or coated and shaved vectors is determined using a flow cytometer (ATTUNE NXT, Thermofisher).

The genomic integration of the transgene delivered with shaved, unshaved EVs or PNPs is quantified by qPCR. HEK293T cells or Jurkat Cells are seeded in 24-well plates at 8×10⁴ cells/well. Cells are transduced with a minimum of 3 successive dilutions of shaved or unshaved EVs and incubated for 72 h at 37° C. and 5% CO₂. After 72 hours, cells are harvested and pellets collected. Total genomic DNA from transduced cell-pellets is extracted using the MAXWELL RSC Blood DNA kit (Promega). Proviral DNA quantification is performed by qPCR using PERFECTA MultiPlex ToughMix (Quantabio). The amplification of integrated sequences is performed using probe and primers specific to the LTR (long terminal repeat sequences).

### Example 8—Coating of EVs with Targeting Agent

Coating of EVs with targeting agent is carried out generally as described in Example 7 except that a mixture of free-polymer and polymer covalently-coupled to the targeting moiety is used Buffer composition is adapted not to impact the solubility of the selected polymers coupled to the targeting agents.

TABLE 3
summarizes characteristics of aptamers used as targeting agents
specific for T lymphocytes or NK cells.

| | | | SEQ ID | Length | Chemistry | Reference/ |
|---|---|---|---|---|---|---|
| Target | ID | Sequence | NO: | (nt) | | source |
| CD3 | ARACD3- | UAUAGACUUUAAUGUCUCA | 8 | 50 | RNA | WO2021019301A2 |
| | 3700006 | UUUUCG | | | | |
| | | CAGCGAUUCUUGUUUAUUU | | | | |
| | | AACAUA | | | | |
| | ARACD3- | UCUAAGCAAUAUUGUUUGC | 9 | 48 | RNA | WO2021019301A2 |
| | 0010209 | UUUUGC | | | | |
| | | AGCGAUUCUGUUUCGAUAU | | | | |
| | | AUUA | | | | |
| | core40t | GGGTTTGGCATCGGGTCTG | 10 | 21 | DNA | WO2021019301A2 |
| | | GC | | | | |
| CD8 | A3t | CCAGAGTGACGCAGCAACA | 11 | 88 | DNA | Kacherovsky |
| | | GAGGTGTAG | | | | et al. Nat. |
| | | AAGTACACGTGAACAAGCTT | | | | Biomed. Eng. |
| | | GAAATTGTC | | | | 2019 10: |
| | | TCTGACAGAGGTGGACACG | | | | 783 |
| | | GTGGCTTTTAGT | | | | |
| | A1 | ATCCAGAGTGACGCAGCAC | 12 | 88 | DNA | Kacherovsky |
| | | GCAGCACCCGT | | | | et al. Nat. |
| | | GGTAGTGTATCAGGGAGAC | | | | Biomed. Eng. |
| | | ACTACGTGATG | | | | 2019 10: |
| | | CAGCTTGAAATGGACACGG | | | | 783 |
| | | TGGCTTAGT | | | | |
| | A3 | ATCCAGAGTGACGCAGCAA | 13 | 88 | DNA | Kacherovsky |
| | | CAGAGGTGTAG | | | | et al. Nat. |
| | | AAGTACACGTGAACAAGCTT | | | | Biomed. Eng. |
| | | GAAATTGTCTC | | | | 2019 10: |
| | | TGACAGAGGTGGACACGGT | | | | 783 |
| | | GGCTTAGT | | | | |
| | A8 | ATCCAGAGTGACGCAGCAG | 14 | 88 | DNA | Kacherovsky |
| | | CTCGATCGTATA | | | | et al. Nat. |
| | | GCCGTGACGCAGCTTGAAA | | | | Biomed. Eng. |
| | | TGGGATCGCG | | | | 2019 10: |
| | | TCCACAGTTTTGGACACGGT | | | | 783 |
| | | GGCTTAGT | | | | |
| NKG2D | 3056- | Undisclosed, part of | | 40 | DNA | US10316321 |
| | 35 | SOMAscan ® assay | | | | |
| | 3056- | Undisclosed, part of | | 40 | DNA | US10316321 |
| | 11 | SOMAscan ® assay | | | | |

### Example 9—Contacting of PNPs with Cells

PNPs with or without targeting agents are put in contact with primary cells to evaluate their ability to transfer the gene of interest by restoring transduction or potentially by transfection.

Contacts with cells lines are performed as described in Example 7 on Jurkat et HEK293T cells.

The contact of PNPs with primary cells is performed on peripheral blood mononuclear cells (PBMCs) or isolated human T and NK cells. Human buffy coats are collected from Etablissement Français du Sang (Rungis, France). PBMCs are purified by FICOLL (Cytiva, FICOLL-PAQUE PLUS) gradient separation. T cells or NK cells are then purified from PBMCs by magnetic isolation using T cells or NK cells negative or positive isolation kits (Miltenyi Biotec) or directly from buffy coats using STRAIGHTFROM kits (Mitenyi Biotec). Cells are then cultured in TEXMACS medium (Miltenyi Biotec, 130-097-196) with or without T cell Transact (Miltenyi Biotec, 130-128-758) for polyclonal stimulation Polymer coated EVs/PNPs are a different MOI (MOI 1, 2, 5, 10, 20, 50 or 100) on PBMCs, isolated T cells or NK cells and cultured at 37° C. and 5% CO₂. GFP expression or CAR expression is assessed by flow cytometry (ATTUNE NXT, Thermofisher) at 3, 7, 10 and 14 days post-transduction to assess the stability of the transgene expression.

Cell pellets are simultaneously collected to quantify from extracted genomic DNA and by qPCR the integration into the cell genome. Altogether, the transgene expression by flow cytometry and qPCR quantification over time allow the characterization of the gene delivery stability (eg. stable transduction or transient transfection).

Moreover, an extensive cell phenotyping is performed by flow cytometry at each time point after contact with coated shaved EVs.

Within PBMCs, viable CD3+T lymphocytes, CD19+B cells, CD56+NK cells, CD3+CD56+NKT cells, CD16+/SSChigh Neutrophils and CD16-/SSChigh eosinophils, CD14+Monocytes (including classical, intermediate and non-classical subsets) are characterized for their proportion and their GFP or CD19 CAR expression level. The specificity of contact of the coated EVs/PNPs with targeting agent is compared with the coated EVs/PNPs without targeting agent.

The phenotype of isolated T cells expressing the transgene is studied with the detection of the CD4+ T cells, CD8+ T cells, Treg cells, and TCRgamma/delta T lymphocytes. Among CD4+ and CD8+ T cell subpopulations, naïve, memory, effector memory and central memory cells are characterized using antibodies specific to CD45RA, CD62L, CCR7, CD27, CD45RO and CD127. Activation markers such as CD25, CD30, CD69, CD137 and CD154 are detected and the exhaustion profile after contact of the coated EVs/PNPs is evaluated using antibodies specific to PD1, CTLA4, TIM3, LAG3 and TIGIT.

Isolated NK cells expressing GFP or CD19 CAR are detected using their expression of CD56+ and lack of expression of CD3+. NK phenotyping to evaluate their activation and exhaustion profile is performed with a series of markers including but not limiting to NKG2A, NKG2D, NKp30, NKp44, NKp46, CD11a, CD11c, CD16, CD25, CD28, CD54 (ICAM-1) CD69, CD226, DNAM-1, TIM3, PD1.

Finally, human cytokines secreted by the PBMCs, isolated T cells or NK cells after 3, 7, 10 and 14 days in culture will be quantified on cell culture supernatant using LEGENDPLEX kits from Biolegend.

### REFERENCES

Du R., Wang C., Zhu L. and Yang Y. Extracellular Vesicles as Delivery Vehicles for Therapeutic Nucleic Acids in Cancer Gene Therapy: Progress and Challenges, Pharmaceutics, 2022, 14:2236
Teng F. and Fussenegger M. Shedding Light on Extracellular Vesicle Biogenesis and Bioengineering, Advanced Science, 2021, 8:2003505
Joshi B. S., Ortiz D. and Zuhorn I. S. Converting extracellular vesicles into nanomedicine: loading and unloading of cargo, Materials Today Nano, 2021, 16:100148
Rezaie J., Feghhi M. & Etemadi T. A review on exosomes application in clinical trials: perspective, questions, and challenges, Cell Communication and Signaling, 2022, 20:145
Rheinemann L. and Sundquist W. I. Virus Budding, Encyclopedia of Virology, 4^{th} Edition, 2021, 519-528
Coffin J. M., Hughes S. H and Varmus H. E. Retroviruses, Cold Spring Harbor Laboratory Press, 1997
Bell N. M. and Lever A. M. L. HIV Gag polyprotein: processing and early viral particle assembly, Trends in Microbiology, 2013, 21:136-44
Dautzenberg I. J. C, Martijn J. W. E. and Hoeben R. C. The stability of envelope-pseudotyped lentiviral vectors, Gene Therapy, 2021, 28:89-104
Chen J., Li P., Zhang T., Xu Z., Huang X., Wang R and Du. L. Review on Strategies and Technologies for Exosome Isolation and Purification, Frontiers in Bioengineering and Biotechnology, 2022, 9:811971, 1-18
Menon A. P., Moreno B., Meraviglia-Crivelli D., Nonatelli F., Villanueva H., Barainka M., Zheleva A., van Santen H. M. and Pastor F. Modulating T Cell Responses by Targeting CD3, Cancers, 2023, 15:1189
Lian G., Mak T. S. K., Yu X. and Lan H. Y. Challenges and Recent Advances in NK Cell-Targeted Immunotherapies in Solid Tumors, International Journal of Molecular Sciences, 2022, 23:164
Zufferey, R., Nagy D., Mandel R. J., Naldini L. and Trono D. Multiply attenuated lentiviral vector achieves efficient gene delivery in vivo, Nature Biotechnology, 1997, 15:871-875
Sadelain M., Brentjens R. and Rivière I. The basic principles of chimeric antigen receptor design, Cancer Discovery, 2013, 3:388-398
Kloss C. C., Condomines M., Cartellieri M., Bachmann M. and Sadelain M. Combinatorial antigen recognition with balanced signaling promotes selective tumor eradication by engineered T cells, Nature Biotechnology, 2013, 31:71-75
Riccardi C., Napolitano E., Musumeci D. and Montesarchio D. Dimeric and Multimeric DNA Aptamers for Highly Effective Protein Recognition, Molecules, 2020, 25:5227
Levine B. L, Humeau L. M., Boyer J., MacGregor R. R., Rebello T, Lu X., Binder G. K., Slepushkin V., Lemiale F., Mascola J. R., Bushman F. D., Dropulic B. and June C. H. Gene transfer in humans using a conditionally replicating lentiviral vector, PNAS, 2006, 103:17372-17377
Perry C. and Rayat A. C. M. E. Lentiviral Vector Bioprocessing, Viruses, 2021, 13:268

We claim:

1. A mammalian-cell-derived extracellular vesicle (EV) that has been treated with a protease to remove proteins on the outer surface of the EV, wherein the surface of the EV is at least partially coated with at least one polymer selected from the group consisting of polyarginine, polylysine, polyethylene Imine (PEI), chitosan, poly(β-amino ester) (PBAE), PEI-co-polyhistidine copolymer, polylysine dendigraft/dendrimer/branched polymer, star shaped polyaminoacid based polymer, chondroitin sulfate, heparosan, poly glutamic acid (PGA), hyaluronic acid, dextran sulfate, polysarcosine, lipid-polyethyleneglycol (PEG), polycarboxybetaine, polysulfobetaine, and polyphosphorylcholine.

2. The EV of claim 1, wherein the EV does not have any full-length viral envelope proteins on its surface.

3. The EV of claim 1, comprising a retroviral capsid comprising a pair of RNAs encoding a non-lentiviral protein, preferably wherein an RNA encodes a chimeric antigen receptor.

4. (canceled)

5. The EV of claim 1, wherein the EV does not have any full-length CAR or non-lentiviral protein on its surface.

6. The EV of claim 1, wherein the capsid is an HIV-1 capsid or wherein the capsid comprises a reverse transcriptase and/or an integrase.

7. (canceled)

8. The EV of claim 1, wherein the protease is proteinase K.

9. (canceled)

10. (canceled)

11. (canceled)

12. The EV of claim 1, further comprising a CD3 or NKG2D targeting agent.

13. (canceled)

14. (canceled)

15. (canceled)

16. (canceled)

17. A method comprising contacting the EV of claim 1 with a mammalian cell, preferably wherein the cell is a T cell or an NK cell.

18. (canceled)

19. (canceled)

20. A method of modifying the outer surface of mammalian-cell-derived extracellular vesicles (EVs) comprising:
a) purifying EVs from a mammalian cell;
b) treating the EVs with a protease to remove proteins on the outer surface of the EVs; and
c) purifying the treated EVs from the protease, wherein the surface of the EVs is at least partially coated with at least one polymer selected from the group consisting of polyarginine, polylysine, polyethylene Imine (PEI), chitosan, poly(β-amino ester) (PBAE), PEI-co-polyhistidine copolymer, polylysine dendigraft/dendrimer/branched polymer, star shaped polyaminoacid based polymer, chondroitin sulfate, heparosan, poly glutamic acid (PGA), hyaluronic acid, dextran sulfate, polysarcosine, lipid-polyethyleneglycol (PEG), polycarboxybetaine, polysulfobetaine, and polyphosphorylcholine.

21. The method of claim 20, wherein the cell expresses retroviral Gag and Pol proteins.

22. The method of claim 20, wherein the EVs comprise an RNA encoding a non-lentiviral protein.

23. The method of claim 23, wherein the RNA encodes a chimeric antigen receptor.

24. The method of claim 20, further comprising contacting the EVs with a mammalian cell, after the protease treatment.


SEQUENCE LISTING (14 sequences)

SEQ ID NO: 1 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 4 - DNA, 1458 bp, synthetic construct
  source 1..1458: mol_type = other DNA
atggccctgcctgtgacagccctgctgctgcccctggctctcctgctgcatgcggcgcgccccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacaccagccggctccacagcggcgtgcccagcagattttctggcagcggcagcggcaccgactacagcctgaccatctccaacctggaacaggaagatatcgctacctacttctgtcagcaaggcaacaccctgccctacaccttcggcggaggcaccaagctggaaatcaccggcggaggcggaagtggaggtggaggatctggcggcggaggctccgaagtgaagctgcaggaaagcggccctggcctcgtggcccctagccagagcctgtccgtgacctgtaccgtgtccggcgtgtccctgcccgactacggcgtgtcctggatcagacagcctcccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtcgtcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - DNA, 27 bp, synthetic construct
  source 1..27: mol_type = other DNA
aaccattaggagtagcacccaccaagg

SEQ ID NO: 6 - DNA, 20 bp, synthetic construct
  source 1..20: mol_type = other DNA
tggaggaggagatatgaggg

SEQ ID NO: 7 - DNA, 20 bp, synthetic construct
  source 1..20: mol_type = other DNA
ctgctgcactataccagaca

SEQ ID NO: 8 - RNA, 50 nt, synthetic construct
  source 1..50: mol_type = other RNA
tatagactttaatgtctcattttcgcagcgattcttgtttatttaacata

SEQ ID NO: 9 - RNA, 48 nt, synthetic construct
  source 1..48: mol_type = other RNA
tctaagcaatattgtttgcttttgcagcgattctgtttcgatatatta

SEQ ID NO: 10 - DNA, 21 bp, synthetic construct
  source 1..21: mol_type = other DNA
gggtttggcatcgggtctggc

SEQ ID NO: 11 - DNA, 88 bp, synthetic construct
  source 1..88: mol_type = other DNA
ccagagtgacgcagcaacagaggtgtagaagtacacgtgaacaagcttgaaattgtctctgacagaggtggacacggtggcttttagt

SEQ ID NO: 12 - DNA, 88 bp, synthetic construct
  source 1..88: mol_type = other DNA
atccagagtgacgcagcacgcagcacccgtggtagtgtatcagggagacactacgtgatgcagcttgaaatggacacggtggcttagt

SEQ ID NO: 13 - DNA, 88 bp, synthetic construct
  source 1..88: mol_type = other DNA
atccagagtgacgcagcaacagaggtgtagaagtacacgtgaacaagcttgaaattgtctctgacagaggtggacacggtggcttagt

SEQ ID NO: 14 - DNA, 88 bp, synthetic construct
  source 1..88: mol_type = other DNA
atccagagtgacgcagcagctcgatcgtatagccgtgacgcagcttgaaatgggatcgcgtccacagttttggacacggtggcttagt